(19) United States
(12) Patent Application Publication
(10) Pub. No.: US 2012/0252748 A1
(43) Pub. Date: Oct. 4, 2012

(54) METHODS AND COMPOSITIONS FOR DETERMINING THE RESPONSIVENESS OF CANCER THERAPEUTICS

(75) Inventor: Marie Monaco, New York, NY (US)

(73) Assignee: New York University

(21) Appl. No.: 13/432,651

(22) Filed: Mar. 28, 2012

Related U.S. Application Data
(60) Provisional application No. 61/468,410, filed on Mar. 28, 2011.

(51) Int. Cl. G01N 33/574; C12Q 1/68; A61K 31/337; A61K 31/7036; A61P 35/00; A61K 31/55; A61K 31/664; A61K 31/7088; A61K 31/713; G01N 33/577; A61K 31/7068

(52) U.S. Cl. 514/34; 435/7.1; 436/501; 435/6.12; 435/7.9; 435/7.92; 514/449; 514/49; 514/214.02; 514/138; 514/44 R; 514/44 A

(57) ABSTRACT

The present invention relates to methods for predicting the responsiveness of a patient to a breast cancer treatment regimen by assaying a biological sample to determine the level of expression of the long-chain fatty acyl-CoA synthetase 4 (ACSL4) in the biological sample. The present invention also provides ACSL4 inhibitors and uses of ACSL4 inhibitors as adjuvant therapies in breast cancer treatment regimens.

### CROSS REFERENCE TO RELATED APPLICATIONS

The present application claims the benefit of U.S. Provisional Patent Application No. 61/468,410, filed Mar. 28, 2011, the disclosure of which is incorporated by reference herein in its entirety.

### BACKGROUND OF THE INVENTION

1. Field of the Invention

The present invention relates generally to prognostic methods which are useful in medicine, particularly cancer treatment regimens. More particularly, the present application relates to methods of predicting the responsiveness and survivability of a patient to a breast cancer therapeutic by determining the level of a biological marker in a biological sample obtained from the patient.

2. Background of the Invention

Cancer arises when a normal cell undergoes neoplastic transformation and becomes a malignant cell. Transformed (malignant) cells escape normal physiologic controls specifying cell phenotype and restraining cell proliferation. Transformed cells in an individual's body thus proliferate, forming a tumor. When a tumor is found, the clinical objective is to destroy malignant cells selectively while mitigating harm to normal cells in the patient undergoing treatment.

Breast cancer (malignant breast neoplasm) is a malignant proliferative disease which originates from the inner lining of milk ducts (ductal carcinomas) or the lobules (lobular carcinomas) that supply the ducts with milk in breast tissue. Effective methods in diagnosing breast cancer include screening techniques, including mammography and clinical breast exams, ultrasound, magnetic resonance imaging (MRI), positron emission tomography (PET) and biopsy, including fine needle aspiration and cytology (FNAC), core biopsy where a section of the breast lump is removed and excisional biopsy, where the entire lump is removed.

Following the identification of breast cancer in a patient, several tests are conducted to determine the size, stage, and rate of growth of the cancer. Additionally, several assays are performed on biological samples taken from the patient to determine the presence of biological markers such as for example, estrogen receptor (ER), progesterone receptor (PR), androgen receptor, and human epidermal growth factor type 2 receptor (HER2/neu) (also known as ErbB-2) in the cancer in order to determine the most effective treatment regimen and prognosis.

Following diagnosis, breast cancers are usually treated with surgery and then possibly with chemotherapy, radiation or other adjuvant therapies. Hormone-positive cancers, those which test positive for the presence of estrogen and/or progesterone receptors, are treated with long term hormone blocking therapy. Overall, a patient's prescribed treatment regimen is determined according to the prognosis and risk of recurrence.

Stage 1 cancers generally have a more favorable prognosis and are generally treated with lumpectomy and radiation. Breast cancers which are positive for human epidermal HER2/neu receptor are generally treated with trastuzumab (Herceptin). Chemotherapy is generally uncommon for most Stage 1 cancers.

Stage 2 and 3 cancers generally have a progressively poorer prognosis and greater risk of recurrence. Stage 2 and 3 cancers are generally treated with lumpectomy or mastectomy with or without lymph node removal, chemotherapy (plus trastuzumab thr HER2/neu+cancers) and sometimes radiation.

Stage 4, metastatic cancers have a poor prognosis and are managed by various combinations of all available treatments including surgery, radiation, chemotherapy and targeted therapeutics such as hormone blocking therapy.

Although treatment of breast cancer typically involves surgery to remove the breast cancer cells, additional adjuvant therapy is often utilized. Several types of adjuvant therapy are known including radiation, chemotherapy agents such as docetaxel, capecitabine, cyclophosphamide and doxorubicin, hormonal based therapeutic agents including selective estrogen receptor modulators (SERMS), such as tamoxifen and aromatase inhibitors, and biological therapeutic agents including, for example, monoclonal antibodies such as cetuximab (Erbitux®), panitumumab, zalutumumab, nimotuzumab, matuzumab, lapatinib (Tykerb®) or trastuzumab (Herceptin®).

ER and PR status in breast cancer cells is an important prognostic factor. ER and PR tests measure the amount of these hormone receptors in the cancer tissue. Hormone blocking therapy is utilized in instances where the breast cancer tests positive for the presence of estrogen receptors (ER⁺) and progesterone receptors (PI⁺). Breast cancers cells containing ER and/or PR are known as hormone-receptor positive cells whereas cells with normal or low levels of these receptors are referred to as hormone-receptor negative cells. Approximately 75% of breast cancers are estrogen receptor-positive (ER⁺). Additionally, about 65% of ER⁺ breast cancer cells are also progesterone receptor positive (PR⁺). Breast cancer cells expressing ER and PR are likely to respond to hormonal-based treatments. Generally, the absence of ER in a cancer cell, referred to as hormone independence (HI), is correlated with poor prognosis, an increased incidence of recurrence of breast cancer following treatment and an increased incidence of metastatic disease. Additionally, the presence of the androgen receptor in a biological sample has also been linked to the predicted effectiveness of hormonal based therapies. Tumors lacking ER and PR will likely only respond to adjuvant therapy involving chemotherapy.

The HER2/neu receptor status is also an important prognostic factor in treating breast cancer patients. A second type of adjuvant therapy utilizes a monoclonal antibody specific for breast cancer cells which have an amplification of the HER2/neu gene or overexpression of the HER2/neu receptor. Consequently, breast cancer cells are also routinely tested for overexpression of HER2/neu due to its prognostic role. In general, assay results showing the presence of more HER2/neu genes or a higher expression level of HER2/neu protein as compared to normal cells indicates a poor prognosis and an association with increased disease recurrence. Approximately 30% of breast cancers have an amplification of the HER2/neu gene or overexpression of its protein product. Overexpression of the HER2/neu gene can be suppressed by the amplification of other genes and the use of monoclonal antibodies specific for the HER2/neu receptor, for example, trastuzumab (Herceptin®). Trastuzumab is effective only in breast cancer where the HER2/neu receptor is overexpressed.

Acyl-CoA synthetase long-chain family member 4 (ACSL4, ACS4, FACL4, LACS4, MRX63, MRX68) (OMIM: 300157, MGI: 1354713, HomoloGene: 56282, GeneCards: ACSL4 Gene, GenBank Accession No. AF030555) is an essential enzyme involved in lipid biosynthesis and fatty acid degradation, Cao et al. Genomics 49, 327-330 (1998), which is incorporated by reference herein in its entirety. ACSL4 is expressed in the human placenta, brain, testes, ovary, spleen, and adrenal cortex and to a lesser extent in the gastrointestinal system, including liver. ACSL4 expression was also found to be highly elevated in colon adenocarcinoma and hepatocellular carcinoma (HCC) compared with the normal adjacent tissue. Can et al., Cancer Res. 61, 8429-8434 (2001), Liang et al. World J. Gastroenterol. 11(17):2557-2563 (2005), the disclosures of each of which are incorporated by reference herein in their entireties. Accordingly, the detection of ACSL4 could be a potentially important biological marker for the early onset of colon adenocarcinoma and hepatocellular carcinoma.

As previously stated, the use of hormonal and biological therapeutic agents as neoadjuvant or adjuvant breast cancer treatment therapies is dependent on the presence of certain biological markers. Biological markers are typically proteins found in biological samples, including, for example, blood, urine, or tissue samples when cancer is present. With regard to hormonal therapy, the effectiveness of the agent depends on the detection of estrogen (ER⁺) and/or progesterone (PR⁺) receptors in a biological sample. With respect to biological therapeutic agents, it has been shown, for example, that the effectiveness of the monoclonal antibody trastuzumab is dependent upon the presence of the growth-promoting HER2/neu protein.

Breast cancer cells without estrogen receptors (ER⁻), progesterone receptors (PR−), and large amounts of HER2/neu protein (HER2/neu-negative) are referred to as “triple-negative” breast cancer (NBC). Breast cancer cells without estrogen receptors (ER⁻), progesterone receptors (PR⁻), androgen receptors (AR⁻) and large amounts of HER2/neu protein (HER2/neu-negative) are, for the purposes of this application, referred to as “quadruple-negative” breast cancer (NBC).

In order to determine the presence of estrogen receptors, progesterone receptors, androgen receptors and HER2/neu, a biological sample is typically taken from the patient and the DNA, RNA, or protein is measured, for example, using an immunohistochemistry (IHC) or fluorescence in-situ hybridization (FISH) assay. To determine whether a hormonal based adjuvant therapy or use of a HER2/neu monoclonal antibody would be effective, four separate assays on the biological sample to measure estrogen receptors, progesterone receptors, androgen receptors and HER2/neu, would need to be performed to determine whether the breast cancer cells have estrogen receptors, progesterone receptors, androgen receptors and HER2/neu protein.

Consequently, it would be beneficial to have a single assay capable of determining triple-negative and quadruple negative breast cancer types to make a determination as to the patient's optimal cancer treatment regimen as well as the predicted responsiveness of a patient to a hormonal or biological based cancer therapeutic.

### SUMMARY OF THE INVENTION

The needs identified above are met by the present invention providing methods for predicting the responsiveness of a patient to a breast cancer treatment regimen by assaying a biological sample for expression of long-chain fatty acyl-CoA synthetase 4 (ACSL4, also referred to as ACS4, FACL4, LACS4, MRX63 and MRX6). The present invention also provides ACSL4 inhibitors and uses of ACSL4 inhibitors as neoadjuvant or adjuvant therapies in breast cancer treatment regimens.

In an aspect of the invention, methods are provided for predicting the responsiveness of a patient to a breast cancer treatment regimen comprising obtaining a biological sample from the patient, assaying the biological sample for expression of ACSL4, quantitating the level of ACSL4 expression in the biological sample, comparing the level of ACSL4 in the biological sample to the level of ACSL4 expression in a control sample or comparing the level of ACSL4 expression in the biological sample to a predetermined threshold level, and determining that the patient is responsive to a cancer treatment regimen where the level of ACSL4 expression in the biological sample is less than the level of ACSL4 expression in the control sample or less than the predetermined threshold level or determining that the patient is not responsive to a cancer treatment regimen where the level of ACSL4 expression in the biological sample is greater than the level of ACSL4 expression in the control sample or greater than the predetermined threshold level.

In one embodiment of any of the aspects of the present invention, the biological sample is a tumor biopsy obtained by fine needle aspiration and cytology (FNAC), core biopsy or an excisional biopsy.

In one embodiment of any of the aspects of the present invention, the assaying of the biological sample for expression of the long-chain fatty acyl-CoA synthetase 4 (ACSL4) is performed by detection of the ACSL4 protein using immunohistochemistry (IHC) or western blot. In a further embodiment of any of the aspects of the present invention, the assaying of the biological sample for expression of the long-chain fatty acyl-CoA synthetase 4 (ACSL4) is performed by detection of ACSL4 mRNA from the biological sample using reverse-transcriptase polymerase chain reaction (RT-PCR).

In one embodiment of any of the aspects of the present invention, the immunohistochemistry assay is performed using a detectably labeled antibody specific for ACSL4. In a further embodiment of any of the aspects of the present invention, the antibody is a monoclonal antibody. In a further exemplary embodiment, the antibody is conjugated to an enzyme, such as, for example, alkaline phosphatase (AP) and horseradish peroxidase (HRP), or the antibody is labeled with a chromagen car fluorophore. Fluorophores which can be used in the assay are known in the art and include, but are not limited to, fluorescein or rhodamine.

In one embodiment of any of the aspects of the present invention, the breast cancer treatment regimen is a hormonal-based therapy comprising a selective estrogen receptor modulator (SERM), a selective estrogen receptor down-rezulator (SERD) or an aromatase inhibitor. Exemplary SEMIS comprise tamoxifen, raloxifene, toremifene, or lasofoxifene. Exemplary SERBS include fulvestrant. Exemplary aromatase inhibitors include anastrozole, letrozole, exemestane, vorozole, formestane or fadrozole.

In one embodiment of any of the aspects of the present invention, the breast cancer treatment regimen is a receptor tyrosine kinase inhibitor. In an exemplary embodiment, the receptor tyrosine kinase inhibitor is an antibody, preferably a monoclonal antibody. In one embodiment of any of the aspects of the present invention, the monoclonal antibody is directed against the epidermal growth factor receptor (EGFR), erythroblastosis group B (ErbB) receptor tyrosine kinase, or HER2/neu. Exemplary embodiments of receptor tyrosine kinase inhibitors comprise cetuximab, panitumumab, zalutumumab, nimotuzumab, matuzumab, or trastuzumab. In one embodiment of any of the aspects of the present invention, the receptor tyrosine kinase inhibitor is a small molecule inhibitor selected from the group comprising gefitinib, imatinib, erlotinib, lapatinib, canertinib, sunitinib, vandetanib, vatalanib, sorafenib and leflunomide. In a preferred embodiment of any of the aspects of the present invention, the monoclonal antibody is directed against the HER2/neu receptor.

In one embodiment of any of the aspects of the present invention, the breast cancer treatment regimen is an androgen receptor inhibitor. In an exemplary embodiment, the androgen receptor inhibitor targets the androgen receptor protein. In an exemplary embodiment, the inhibitor of the androgen receptor protein is an antibody, preferably a monoclonal antibody specific to the androgen receptor. In one embodiment of any of the aspects of the present invention, the androgen receptor inhibitor is a small molecule inhibitor. In a further exemplary embodiment, the androgen receptor inhibitor is a nucleic acid inhibitor. In an exemplary embodiment, the nucleic acid inhibitor is an siRNA, dsRNA or an enzymatic nucleic acid.

In an aspect of the invention, methods are provided for determining a breast cancer adjuvant treatment regimen for a cancer patient comprising obtaining a biological sample from the patient, assaying the biological sample for expression of ACSL4, quantitating the level of ACSL4 expression in the biological sample, comparing the level of ACSL4 expression in the biological sample to the level of ACSL4 expression in a control sample or comparing the level of ACSL4 expression in the biological sample to a threshold, and determining a suitable breast cancer adjuvant treatment regimen depending on whether the level of ACSL4 expression in the biological sample is less than or greater than the level of ACSL4 expression in the control sample or less than or greater than the predetermined threshold level.

In an aspect of the invention, methods are provided for identifying breast cancers lacking expression of estrogen receptor (ER⁻), progesterone receptor (PR⁻), and human epidermal growth factor 2 (HER2/neu⁻), collectively referred to as triple-negative breast cancers (TNBC), comprising obtaining a biological sample from the patient, assaying the biological sample for expression of ACSL4, quantitating the level of ACSL4 expression in the biological sample and comparing the level of ACSL4 expression in the biological sample to the level of ACSL4 expression in a control sample or comparing the level of ACSL4 expression in the biological sample to a threshold level, and determining that the breast cancer in the patient is TNBC where the level of ACSL4 expression in the biological sample is higher than the level of ACSL4 expression in the control sample or higher than the threshold or determining that the breast cancer in the patient is not TNBC where the level of ACSL4 expression in the biological sample is lower than the level of ACSL4 expression in the control sample or lower than the predetermined threshold level.

In one aspect of the invention, methods are provided for identifying breast cancers lacking expression of estrogen receptor (ER⁻), progesterone receptor (PR⁻), human epidermal growth factor 2(HER2/neu⁻), and androgen receptor (AR⁻), collectively referred to as quadruple-negative breast cancers (QNBC), comprising obtaining a biological sample from the patient, assaying the biological sample for expression of ACSL4, quantitating the level of ACSL4 expression in the biological sample and comparing the level of ACSL4 expression in the biological sample to the level of ACSL4 expression in a control sample or comparing the level of ACSL4 expression in the biological sample to a predetermined threshold level, and determining that the breast cancer in the patient is QNBC where the level of ACSL4 expression in the biological sample is higher than the level of ACSL4 expression in the control sample or higher than the threshold or determining that the breast cancer in the patient is not QNBC where the level of ACSL4 expression in the biological sample is lower than the level of ACSL4 expression in the control sample or lower than the predetermined threshold level.

In one aspect of the invention, methods are provided for identifying estrogen and/or androgen insensitive breast cancers, comprising assaying a biological sample for expression of ACSL4, quantitating the level of ACSL4 expression in the biological sample and comparing the level of ACSL4 expression in the biological sample to the level of ACSL4 expression in a control sample or comparing the level of ACSL4 expression in the biological sample to a predetermined threshold level, and determining that the breast cancer is estrogen and/or androgen insensitive where the level of ACSL4 expression in the biological sample is higher than the level of ACSL4 expression in the control sample or higher than the predetermined threshold level or determining that the breast cancer in the patient is not estrogen and/or androgen insensitive where the level of ACSL4 expression in the biological sample is lower than the level of ACSL4 expression in the control sample or lower than the predetermined threshold level.

In an aspect of the invention, methods are provided for treating a patient with breast cancer comprising the use of an ACSL4 inhibitor. In one embodiment of this aspect of the invention, the breast cancer lacks expression of estrogen receptor (ER), progesterone receptor (PR⁻), human epidermal growth factor 2 (HER2/neu), and/or androgen receptor (AR⁻). In one embodiment of this aspect of the invention, the breast cancer is estrogen and/or androgen insensitive. In one embodiment of this aspect of the invention, the breast cancer is a triple-negative breast cancer (TNBC) or a quadruple-negative breast cancer (QNBC).

In one embodiment of this aspect of the invention, the ACSL4 inhibitor targets the ACSL4 protein. In one embodiment of this aspect of the invention, the ACSL4 inhibitor is a small molecule inhibitor. In an exemplary embodiment, the ACSL4 inhibitor is triacin-C or rosiglitazone maleate (Avandia®). In a further exemplary embodiment, the ACSL4 inhibitor is a nucleic acid inhibitor. In an exemplary embodiment, the nucleic acid inhibitor is a small interfering (siRNA), double-stranded (dsRNA), microRNA (miRNA), antisense RNA, aptamer, ribozyme, or an enzymatic nucleic acid. In one embodiment of this aspect of the invention, the ACSL4 inhibitor is administered in combination with a chemotherapeutic agent. In an exemplary embodiment the chemotherapeutic agent is an anthracycline, taxane, cyclophosphamide, capecitabine, vinorelbine, or gemcitabine.

### BRIEF DESCRIPTION OF FIGURES

FIG. 1 compares ACSL4 mRNA expression levels in ER-negative (shaded bars) versus ER-positive (unshaded bars) human breast tumors from ten independent gene expression profile data sets.

FIG. 2 shows the results of the relationship between ACSL4 and ER gene expression levels from 34 ER-negative and 213 ER-positive human breast tumors.

FIG. 3A shows the expression data of the five known isoforms of ACSL (1, 3, 4, 5, and 6) in 19 ER-positive (unshaded bars) and 31 ER-negative (shaded bars) breast cancer cell lines described in Example 2.

FIG. 3B shows the ranges of ACSL4 expression data in the 19 ER-positive and 31 ER-negative breast cancer cell lines described in Example 2.

FIG. 4 shows an immunoblot analyses of ACSL1 and ACSL4 protein expression relative to those of β-actin from ER-positive (MCF-7, MDA-MB-415 and T47D) and ER-negative (BT20, MDA-MB-231, and SKBR3) breast cancer cell lines.

FIG. 5 shows growth response (protein/well (% of control) of the MDA-MB-415 and MCF-7 cell lines in the presence of estrogen (F) compared to the cells grown in the absence of estrogen (control (C) cell line).

FIG. 6A shows the growth curve of MCF-7 control cells in the presence of estrogen compared to estrogen-free conditions.

FIG. 6B shows the growth curves of MCF-7 cells genetically manipulated to express ACSL4 in the presence of estrogen compared to estrogen-free conditions.

FIG. 7A is an immunoblot for ACSL4 and p-actin after a 48 hour transfection with no addition of ACSL4-siRNA (−), control siRNA (C), or addition of ACSL4-siRNA (+).

FIG. 7B shows the cellular growth curve of MDA-MB-231 cells after 48 hours of treatment with control siRNA or ACSL4-siRNA.

FIG. 7C shows the cell growth effects of triacsin C on relative cell number after 48 hours of treatment with control siRNA, or ACSL4-siRNA.

FIG. 8A shows the relationship between AR and ACSL4 mRNA expression in 77 ER-negative breast tumor samples.

FIG. 8B is an immunoblot showing the expression of ACSL4 and β-actin in prostate cancer cell lines which were AR-negative (PC3 and DU145), AR-positive (LNCaP), and AR-positive but androgen-independent for growth.

FIG. 8C shows the relationship between AR and ACSL4 mRNA expression in 98 human prostate tumor samples.

FIG. 9 shows the relationship between HER2 and ACSL4 mRNA expression in 50 breast cancer cell lines.

FIG. 10 shows the nucleotide sequence for acyl-CoA synthetase long-chain family member 4 (ACSL4), transcript variant 1. (SEQ ID NO:1)

FIG. 11 shows the encoded amino acid sequence for acyl-CoA synthetase long-chain family member 4 (ACSL4), transcript variant 1, (SEQ ID NO:2)

FIG. 12 shows the nucleotide sequence for acyl-CoA synthetase long-chain family member 4 (ACSL4), transcript variant 2, (SEQ ID NO:3)

FIG. 13 shows the encoded amino acid sequence for acyl-CoA synthetase long-chain family member 4 (ACSL4), transcript variant 2. (SEQ ID NO:4)

FIG. 14A-B shows ACSL4 expression as a function of hormone/growth factor receptor status in breast cancer cells lines based on data taken from microarray studies.

FIG. 15 shows that expression of ACSL4 in MCF-7 cells results in hormone resistance.

FIG. 16 shows the effect of ACSL4 expression on Tamoxifen sensitivity.

FIG. 17A-B shows the relationship between ACSL4 and HER2 expression in breast cancer cell lines (A) and tumor samples (B).

FIG. 18 shows in Panels AD the expression data for proteins whose expression correlates with sensitivity to trastuzumab, while Panels E-F show expression data for proteins whose expression correlates with trastuzumab resistance.

### DETAILED DESCRIPTION OF PREFERRED EMBODIMENTS

It should be appreciated that the particular implementations shown and described herein are examples and are not intended to otherwise limit the scope of the application in any way.

The published patents, patent applications, websites, company names, and scientific literature referred to herein are hereby incorporated by reference in their entirety to the same extent as if each was specifically and individually indicated to be incorporated by reference. Any conflict between any reference cited herein and the specific teachings of this specification shall be resolved in favor of the latter, likewise, any conflict between an art understood definition of a word or phrase and a definition of the word or phrase as specifically taught in this specification shall be resolved in favor of the latter.

As used in this specification, the singular forms “a,” “an” and “the” specifically also encompass the plural forms of the terms to which they refer, unless the content clearly dictates otherwise. The term “about” is used herein to mean approximately, in the region of roughly, or around. When the teun “about” is used in conjunction with a numerical range, it modifies that range by extending the boundaries above and below the numerical values set forth. In general, the term “about” is used herein to modify a numerical value above and below the stated value by a variance of 20%.

Technical and scientific terms used herein have the meaning commonly understood by one of skill in the art to which the present application pertains, unless otherwise defined. Reference is made herein to various methodologies and materials known to those of skill in the art. Standard reference works setting forth the general principles of recombinant DNA technology include Sam brook el al., “Molecular Cloning: A Laboratory Manual,” 2nd Ed., Cold Spring Harbor Laboratory Press, New York (1989); Kaufman et al., Eds., “Handbook of Molecular and Cellular Methods in Biology in Medicine,” CRC Press, Boca Raton (1995); and McPherson, Ed., “Directed Mutagenesis: A Practical Approach,” IRL Press, Oxford (1991), the disclosures of each of which are incorporated by reference herein in their entireties.

ACSL4, which is also referred to as ACS4, FACL4, LACS4, MRX63 and MRX6, is a lipid metabolic enzyme encoded in humans by the ACSL4 gene. ACSL4 is an isozyme of the long-chain fatty acid-coenzyme A ligase family that converts free long-chain fatty acids into fatty acyl-CoA esters and thereby plays a key role in lipid biosynthesis and fatty acid degradation. The absence of ACSL4 has been correlated with mental retardation, Alport syndrome and elliptocytosis. Additionally, expression of ACSL4 has been recently shown to be increased in colon adenocarcinoma and hepatocellular carcinoma (Cao et al., Cancer Res, 61: 8429-4434; Cao et al., Exp. Mal Med. 39(4): 477-482).

Surprisingly, it was discovered in the present invention that cancer cells, including breast cancers cells, differentially express ACSL4. Importantly, it was discovered in the present invention that ACSL4 has an increased mRNA and/or protein expression level in ER⁻, PR⁻, and AR⁻ breast cancer cells. See Monaco et al., Translational Oncology, 3(2):76-83 (April 2010), which is hereby incorporated by reference in its entirety. Accordingly, breast cancer cells that express high levels of ACSL4 mRNA and/or protein compared to a matching non-malignant control or predetermined threshold level are likely to be insensitive to hormonal-based treatment regimens. Conversely, those breast cancer cells expressing low amounts of ACSL4 mRNA and/or protein compared to a matching non-malignant control or predetermined threshold level are likely to be sensitive to hormonal-based treatment regimens. Additionally, it was also unexpectedly discovered that the detection of an increased ACSL4 mRNA and/or protein expression level in breast cancer cells is indicative of estrogen independent growth and/or estrogen insensitivity, even in instances where the breast cancer cells are ER+. Similarly, it was also surprisingly discovered that the detection of high ACSL4 mRNA and/or protein expression levels in breast cancer cells is indicative of androgen independent growth and/or androgen insensitivity, even in instances where the breast cancer cells are AR+.

It was further surprisingly discovered in the present invention, that ACSL4 mRNA and/or protein expression levels are negatively correlated with expression of the HER2/neu receptor. Accordingly, breast cancers cells expressing high levels of ACSL4 mRNA and/or protein compared to a matching non-malignant control or predetermined threshold level are likely to be negative for HER2/neu receptor and therefore insensitive to monoclonal antibiotic therapeutics directed to the HER2/neu receptor while breast cancers cells expressing low levels of ACSL4 mRNA and/or protein compared to a matching non-malignant control or predetermined threshold level are likely to be negative for HER2/neu receptor and therefore insensitive to monoclonal antibiotic therapeutics directed to the HER2/neu receptor.

The invention provides a method for predicting the responsiveness of a patient to a cancer treatment regimen. The methods suitably comprise obtaining a biological sample from a patient, assaying the biological sample for the expression of long-chain fatty acyl-CoA synthetase 4 (ACSL4), determining the level of ACSL4 expression in the biological sample and comparing the level of ACSL4 expression in the biological sample to the level of ACSL4 expression in a control sample or comparing the level of ACSL4 expression in the biological sample to a predetermined threshold level. In further embodiments of any of the described methods, the patient is determined to be responsive to a cancer treatment regimen where the level of ACSL4 expression in the biological sample is less than the level of ACSL4 expression in the control sample or less than the predetermined threshold level or is determined to not be responsive to a cancer treatment regimen where the level of ACSL4 expression in the biological sample is greater than the level of ACSL4 expression in the control sample or greater than the predetermined threshold level.

In one embodiment, the cancer treatment regimen is a breast cancer treatment regimen. In an exemplary embodiment, the cancer treatment regimen is a hormonal-based therapy, Hormonal-based therapies are known to a person of skill in the art and include, but are not limited to, selective estrogen receptor modulators (SERMs), selective estrogen receptor down-regulator (SERDs), and aromatase inhibitors. Exemplary SERMs include, for example, tamoxifen, raloxifene, toremifene, or lasofoxifene. Exemplary SERDs include, for example, fulvestrant. Exemplary aromatase inhibitors include, for example, anastrozole, letrozole, exemestane, vorozole, formestane or fadrozole.

In one embodiment, the breast cancer treatment regimen is an androgen receptor inhibitor. In an exemplary embodiment, the androgen receptor inhibitor targets the androgen receptor protein. In another exemplary embodiment, the inhibitor of the androgen receptor protein is an antibody, preferably a monoclonal antibody specific to the androgen receptor. In one embodiment of any of the aspects of the present invention, the androgen receptor inhibitor is a small molecule inhibitor. In a further exemplary embodiment, the androgen receptor inhibitor is a nucleic acid inhibitor. In one exemplary embodiment, the nucleic acid inhibitor is an siRNA, ds NA or an enzymatic nucleic acid.

In one embodiment, the breast cancer treatment regimen is a receptor tyrosine kinase inhibitor. In an exemplary embodiment, the receptor tyrosine kinase inhibitor is an antibody, preferably a monoclonal antibody. In another embodiment, the monoclonal antibody is directed against the epidermal growth factor receptor (EGFR), erythroblastosis group B (ErbB) receptor tyrosine kinase, or HER2/neu, Monoclonal antibodies useful as receptor tyrosine kinase inhibitors are known to a person of skill in the art and include, but are not limited to, cetuximab, panitumumab, zalutumumab, nimotuzumab, matuzumab, or trastuzumab. In a preferred embodiment, the monoclonal antibody is directed against the HER2/neu receptor.

In another embodiment, the receptor tyrosine kinase inhibitor is a small molecule inhibitor. Small molecule receptor kinase inhibitors are known to a person of skill in the art and include, but are not limited to, gefitinib, imatinib, erlotinib, lapatinib, canertinib, sunitinib, vandetanib, vatalanib, sorafenib and leflunomide.

A “biological sample” as defined herein refers to tissues, cells, biological fluids and isolates thereof, isolated from a patient, as well as tissues, cells and biological fluids present within a patient. Preferably, the biological sample comprises cells, preferably tumor cells that are isolated from a biospecimen including, but not limited to, blood and blood fractions, saliva, feces, urine, lymph fluid, biopsies, etc. or cells or tissue which have been removed from any part of the body. Preferable, the biological sample contains breast tissue or metastasized breast cancer cells.

In performing the method of this embodiment of the present invention, tumor cells are preferably isolated from the patient. Solid or lymphoid tumors or portions thereof are surgically resected from the patient or obtained by routine biopsy, in a preferred embodiment of any of the described methods, the biological sample is a tumor biopsy obtained by fine needle aspiration and cytology (FNAC), core biopsy or an excisional biopsy. Pre-chemotherapy treatment tumor biopsies are usually available only as fixed paraffin embedded (FPE) tissues, generally containing only a very small amount of heterogeneous tissue. Such FPE samples are readily amenable to micro-dissection, so that ACSL4 gene and protein expression may be determined in tumor tissue uncontaminated with non-malignant stromal tissue. Additionally, comparisons can be made between non-malignant stromal and tumor tissue within a biopsy tissue sample, since such samples often contain both types of cells.

A “control sample” as defined herein refers to a matching non-malignant sample of non-cancerous tissue derived from the same patient as the tumor sample to be analyzed for differential ACSL4 expression. Preferably, a matching non-malignant sample is derived from the same organ as the organ from which the tumor sample is derived. Most preferably, the matching non-malignant tumor sample is derived from the same organ tissue layer from which the tumor sample is derived. Also, it is preferable to take a matching non-malignant tissue sample at the same time a tumor sample is biopsied. In a preferred embodiment, a biological sample is taken from the breast tumor and non-malignant breast tissues taken from the greatest distance from the tumor as possible as clinically appropriate under the circumstances.

A “predetermined threshold level”, “threshold level,” or “threshold” are used interchangeably herein as defined herein relating to ACSL4 expression, refers to a level of differential ACSL4 expression above which (i.e., higher than which), tumors are likely to be insensitive to a hormonal-based treatment regimen, a monoclonal antibody therapeutic directed to the HER2/neu receptor and/or a androgen receptor therapeutic. In one embodiment, the predetermined threshold level is about 1×, about 2×, about 3× the level as compared to an internal control protein or nucleic acid. In an exemplary embodiment, the internal control protein or nucleic acid is β-actin. Example 3 represents the establishment of a “threshold/threshold level/predetermined threshold level,” in accordance with embodiments herein.

### Detection and Quantitation of ACSL4

“Assaying the level of ACSL4 expression” as defined herein refers to a number of known methodologies that may be employed for the detection and/or quantitation of the amount of ACSL4 protein and/or mRNA expression in a biological sample. Furthermore, it should be readily appreciated that the methods disclosed in the present application represent exemplary embodiments for which one of skill in the art would be able to readily determine operative and optimal assay conditions through routine experimentation.

Protein Based Assays

In embodiments, methods for the detection and/or quantitation of ACSL4 protein expression in a biological sample include, but are not limited to, immunohistochemistry (IHC), western blots, ELISA, immunoprecipitation, immunofluorescence, radioimmunoassay (RIA) and flow cytometry.

The protein expression level of ACSL4 in a biological sample may be determined by immunohistochemical staining cells in the sample using a detectably-labeled agent (e.g., an antibody) specific for ACSL4. In a preferred embodiment, the agent is a monoclonal antibody and the detectable label is a chromagen or a fluorophore.

ACSL4 protein expression in a biological sample can be separately detected using a specific agent, most preferably a monoclonal antibody, that is itself detectably labeled, or using an unlabeled antibody specific ACSL4 and a second antibody that is detectably labeled and recognizes the unlabeled antibody specific for ACSL4. Alternatively, any molecule that can be detectably labeled and that specifically binds to ACSL4, can be used in the practice of the methods of the present invention.

The antibodies may be incubated with the sample for a time to form complexes if the ACSL4 is present. The complexes are then visualized by treating the sections with a stain including, for example, diaminobenizidine (DAB) stain under appropriate conditions. In a second step, the tissue may be counterstained with another optical enhancement factor, for example ethyl green. Although a staining technique using peroxidase and ethyl green is exemplary, other stains and optical enhancement factors are also suitable such as alkaline phosphatase based with specific chromagens such as Fast Red, Fast Green, etc.

Following immunohistochemical staining, the optical image of the tissue or cell sample generated by a computer-aided image analysis system may then be magnified under a light microscope and separated into a pair of images. Such equipment can include a light or fluorescence microscope, an image-transmitting camera and a view screen, most preferably also comprising a computer that can be used to direct the operation of the device and store and manipulate the information collected, most preferably in the form of optical density of certain regions of a stained tissue preparation.

Image analysis devices useful in the practice of this disclosure include but are not limited to the CAS 200 (Becton Dickenson, Mountain View, Calif.), Chromavision or Tripath systems. The separated images are enhanced using a pair of optical filters, one having a maximum absorption corresponding to the stain and the other having a maximum absorption corresponding to the counterstain. In an embodiment, a plurality of image analysis filters are used to detect, differentiate, and quantitate the level of staining of different cellular proteins in various components (e.g., membrane, cytoplasm, and nucleus).

In preferred embodiments, specific staining for ACSL4 may be detected, measured and quantitated using image analysis equipment, defined herein as comprising a light or fluorescence microscope, an image transmitting camera and a view screen, most preferably also comprising a computer that can be used to direct the operation of the device and also store and manipulate the information collected, most preferably in the form of optical density of certain regions of a stained tissue preparation. Image analysis devices useful in the practice of this disclosure include, but are not limited to, the CAS 200 system (Becton Dickenson, Mountain View, Calif.). From a digitized image, a nuclear or cytoplasmic image mask is formed by forming the image at one wavelength of light such as red wavelength or green optical filter. The tissue mask may be stored and a second filter is used to form another filtered image of the areas with the optical enhancement factor. Differentiation of cellular characteristics can be made by comparing the first image with the second image to obtain a quantification of material stained with the optical enhancement factor and thus, an assay of the amount of the particular target under study.

After immunohistochemical staining, a quantified measure of the percentage of cells expressing ACSL4 can be taken by digitizing microscope images of stained samples, and converting light intensity values in each picture element (pixel) of the digitized image to optical density values, which correspond to the percentage of stained cell nuclei. In particular, computerized image analysis can be used to determine from a digital grey scale image, a quantity of cells having a particular stain. The grey scale images are representative of the amount of an optical enhancement factor, such as a chromagen, which binds to a specific target under study and thereby allows optical amplification and visualization of the target.

RNA used Assays

RNA isolated from frozen or fresh tumor samples is extracted from the cells by any of the methods typical in the art, for example, Sambrook, Fischer and Maniatis, Molecular Cloning, a laboratory manual, (2nd ed.), Cold Spring Harbor Laboratory Press, New York, (1989). Preferably, care is taken to avoid degradation of the RNA during the extraction process.

However, tissue obtained from the patient after biopsy is often fixed, usually by formalin (formaldehyde) or gluteraldehyde, for example, or by alcohol immersion. Fixed biological samples are often dehydrated and embedded in paraffin or other solid supports known to those of skill in the art. See Plenet et al., Ann Pathol., 21(1):29-47 (2001), which is hereby incorporated by reference in its entirety. Non-embedded, fixed tissue as well as fixed and embedded tissue may also be used in the present methods. Solid supports for embedding fixed tissue are envisioned to be removable with organic solvents for example, allowing for subsequent rehydration of preserved tissue.

RNA may also be extracted from fixed paraffin embedded (FPE) tissue cells by any of the methods as described in U.S. Pat. No. 6,248,535, which is hereby incorporated by reference in its entirety. As used herein, “FPE tissue” means tissue that has been fixed and embedded in a solid removable support, such as storable or archival tissue samples. RNA may be isolated from an archival pathological sample or biopsy sample which is first deparaffinized. An exemplary deparaffinization method involves washing the parafinnized sample with an organic solvent, such as xylene, for example. Deparaffinized sample can be rehydrated with an aqueous solution of lower alcohol. Suitable lower alcohols include, but are not limited to, methanol, ethanol, propanols and butanols. Deparaffinized samples may be rehydrated with successive washes with lower alcoholic solutions of decreasing concentration, for example. Alternatively, the sample is simultaneously deparaffinized and rehydrated. RNA is then extracted from the sample. For RNA extraction, the fixed or fixed and deparaffinized samples can be homogenized using mechanical, sonic or other means known by one of skill in the art. Rehydrated samples may be homogenized in a solution comprising a chaotropic agent, such as, for example, guanidium thiocyanate, using methods known by one of skill in the art. RNA is then recovered from the chaotropic solution by, for example, phenol chloroform extraction, ion exchange chromatography or size exclusion chromatography. RNA may then be further purified using the techniques of extraction, electrophoresis, chromatography, or precipitation or other suitable techniques.

In embodiments, methods for the detection and/or quantitation of ACSL4 mRNA from a biological sample is preferably carried out using, for example, reverse-transcriptase polymerase chain reaction (RT-PCR) methods common in the art. Other methods of quantifying ACSL4 mRNA include, but are not limited to, the use of molecular beacons and other labeled probes useful in multiplexing PCR. Additionally, the present invention envisages the quantification of ACSL4 via use of a PCR free system employing, for example, fluorescent labeled probes. Most preferably, quantification of ACSL4 and an internal control or house-keeping gene (e.g. β-actin) is done using a fluorescence based real-time detection method (ABI PRISM 7700 or 7900 Sequence Detection System [TaqMan®], Applied Biosystems, Foster City, Calif.) or similar system described by Heid et al., Genome Res 1996 6:986-994 and Gibson et al., Genome Res 1996 6:995-1001. The output of the ABI 7700 (TaqMan® Instrument) is expressed in Ct's or “cycle thresholds”. With the TaqMan® system, a highly expressed gene having a higher number of target molecules in a sample generates a signal with fewer PCR cycles (lower Ct) than a gene of lower relative expression with fewer target molecules (higher Ct).

A “house-keeping” gene or “internal control”, as defined herein is any constitutively or globally expressed gene whose presence enables an assessment of ACSL4 mRNA levels. Such an assessment comprises a determination of the overall constitutive level of gene transcription and a control for variations in RNA recovery. “House-keeping” genes or “internal controls” can include, but are not limited to, the cyclophilin gene, β-actin gene, the transferrin receptor gene, GAPDH gene, and the like. Most preferably, the internal control gene is the β-actin gene as described by Eads et al., Cancer Research 1999 59:2302-2306.

A breast tumor sample that has high differential ACSL4 expression is likely to be triple negative or quadruple negative. An advantage of the present invention is that determination that a breast tumor sample is triple negative or quadruple negative can be made using a single assay to determine the level of ACSL4 rather than three or four individual assays, respectively, to test for the presence of ER, PR, HER2/neu or AR. A high differential ACSL4 expression level is indicative of a breast cancer that is generally more aggressive and more difficult to treat.

### ACSL4 Inhibitors

in one aspect of the invention, methods are provided for treating a patient with breast cancer comprising administering to the patient an ACSL4 inhibitor. In one embodiment of this aspect of the invention, the breast cancer lacks expression of estrogen receptor (ER⁻), progesterone receptor (PR⁻), human epidermal growth factor 2 (HER2/neu⁻), and/or androgen receptor (AR⁻). In one embodiment of this aspect of the invention, the breast cancer is estrogen and/or androgen insensitive. In one embodiment of this aspect of the invention, the breast cancer is a triple-negative breast cancers (TNBC) or a quadruple-negative breast cancer (QNBC).

In one embodiment of this aspect of the invention, the ACSL4 inhibitor targets the ACSL4 protein. In another embodiment of this aspect of the invention, the ACSL4 inhibitor is a small molecule inhibitor.

In an exemplary embodiment, the ACSL4 inhibitor is triacin-C or rosiglitazone maleate (Avandia®). In a further exemplary embodiment, the ACSL4 inhibitor is a nucleic acid inhibitor. In an exemplary embodiment, the nucleic acid inhibitor is a small interfering (siRNA), double-stranded (dsRNA), microRNA (miRNA), antisense RNA, aptamer, ribozyme, or an enzymatic nucleic acid. In one embodiment of this aspect of the invention, the ACSL4 inhibitor is administered in combination with a chemotherapeutic agent. In an exemplary embodiment the chemotherapeutic agent is an anthracycline, taxane, cyclophosphamide, capecitabine, vinorelbine, or gemcitabine.

The term “small interfering RNA,” “siRNA” or “short interfering RNA”, as used herein, refers to any nucleic acid capable of mediating RNAi or gene silencing when processed appropriately by a cell. For example, the siRNA can be a double-stranded polynucleotide molecule comprising self-complementary sense and antisense regions, wherein the antisense region comprises complementarity to a target gene. The siRNA can be a single-stranded hairpin polynucleotide having self-complementary sense and antisense regions, wherein the antisense region comprises complementarity to a target gene. The siRNA can be a circular single-stranded polynucleotide having two or more loop structures and a stem comprising self-complementary sense and antisense regions, Wherein the antisense region comprises complementarity to a target gene, and wherein the circular polynucleotide can be processed either in vivo or in vitro to generate an active siRNA capable of mediating RNAi. The siRNA can also comprise a single stranded polynucleotide having complementarity to a target gene, wherein the single stranded polynucleotide can further comprise a terminal phosphate group, such as a 5′-phosphate (see for example Martinez et al., 2002, Cell., 110, 563-574), or 5′,3′-diphosphate. In certain embodiments, the siRNAs are non-enzymatic nucleic acids that bind to a target nucleic acid and alter the activity of the target nucleic acid. Binding and/or activity of the siRNA may be facilitated by interaction with one or more protein or protein complexes, such as the RNA Induced Silencing Complex (or RISC). In certain embodiments, the siRNAs comprise a sequence that is complementary to a target sequence along a single contiguous sequence of one strand of the siRNA molecule.

Optionally, the siRNAs of the invention contain a nucleotide sequence that hybridizes under physiologic conditions (e.g., in a cellular environment) to the nucleotide sequence of at least a portion of the mRNA transcript for the ACSL4 gene to be inhibited (the “target” gene). The double-stranded RNA need only be sufficiently similar to natural RNA that it has the ability to mediate RNAi. The number of tolerated nucleotide mismatches between the target sequence and the siRNA sequence is no more than 1 in 5 base pairs, or 1 in 10 base pairs, or 1 in 20 base pairs, or 1 in 50 base pairs. Mismatches in the center of the siRNA duplex are most critical and may essentially abolish cleavage of the target RNA. In contrast, nucleotides at the 3′ end of the siRNA strand that is complementary to the target RNA do not significantly contribute to specificity of the target recognition, Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by for example, the Smith-Waterman algorithm as implemented in the BESTFIT software program using default parameters (e.g., University of Wisconsin Genetic Computing Group). Greater than 90%, 95%, 96%, 97%, 98%, or 99% sequence identity, or even 100% sequence identity, between the siRNA and the portion of the target gene is preferred. Alternatively, the duplex region of the RNA may be defined functionally as a nucleotide sequence that is capable of hybridizing with a portion of the target gene transcript under stringent conditions (e.g., 400 mM NaCl, 40 mM PIPES pH 6.4, 1 mM EDTA, 50° C. or 70° C. hybridization for 12-16 hours; followed by washing).

The double-stranded structure of dsRNA, may be formed by a single self complementary RNA strand, two complementary RNA strands, or a DNA strand and a complementary RNA strand. Optionally, RNA duplex formation may be initiated either inside or outside the cell. The RNA may be introduced in an amount which allows delivery of at least one copy per cell. Higher doses (e.g., at least 5, 10, 100, 500 or 1000 copies per cell) of double-stranded material may yield more effective inhibition, while lower doses may also be useful for specific applications, Inhibition is sequence-specific in that nucleotide sequences corresponding to the duplex region of the RNA are targeted for inhibition.

As described herein, the subject siRNAs comprise a duplex region about 19-30 nucleotides in length, about 21-27 nucleotides in length, about 21-25 nucleotides in length, or about 21-23 nucleotides in length. The siRNAs are understood to recruit nuclease complexes and guide the complexes to the target gene transcript by pairing to the specific sequences. As a result, the target gene transcript is degraded by the nucleases in the protein complex. In certain embodiments, the siRNA molecules comprise a 3′ hydroxyl group. In certain embodiments, the siRNA constructs can be generated by processing of longer double-stranded RNAs, for example, in the presence of the enzyme dicer. In one embodiment, the Drosophila in vitro system is used. In this embodiment, dsRNA is combined with a soluble extract derived from Drosophila embryo, thereby producing a combination. The combination is maintained under conditions in which the dsRNA is processed to RNA molecules of about 21 to about 27 nucleotides. The siRNA molecules can be purified using a number of techniques known to those of skill in the art. For example, gel electrophoresis can be used to purify siRNAs. Alternatively, non-denaturing methods, such as non-denaturing column chromatography, can be used to purify the siRNA. In addition, chromatography (e.g., size exclusion chromatography), glycerol gradient centrifugation, affinity purification with antibody can be used to purify siRNAs.

Production of the subject dsRNAs (e.g., siRNAs) can be carried out by chemical synthetic methods or by recombinant nucleic acid techniques. Endogenous RNA polymerase of the treated cell may mediate transcription in vivo, or cloned RNA polymerase can be used for transcription in vitro. As used herein, dsRNA, or siRNA molecules of the application need not be limited to those molecules containing only RNA, but further encompasses chemically-modified nucleotides and non-nucleotides. For example, the dsRNAs may include modifications to either the phosphate-sugar backbone or the nucleoside, e.g., to reduce susceptibility to cellular nucleases, improve bioavailability improve formulation characteristics, and/or change other pharmacokinetic properties. To illustrate, the phosphodiester linkages of natural RNA may be modified to include at least one of a nitrogen or sulfur heteroatom. Modifications in RNA structure may be tailored to allow specific genetic inhibition while avoiding a general response to dsRNA. Likewise, bases may be modified to block the activity of adenosine deaminase. The dsRNAs may be produced enzymatically or by partial/total organic synthesis, any modified ribonucleotide can be introduced by in vitro enzymatic or organic synthesis. Methods of chemically modifying RNA molecules can be adapted for modifying dsRNAs (see, e.g., Heidenreich et al. (1997) Nucleic Acids Res, 25:776-780; Wilson et al, (1994) J Mol Recog 7:89-98; Chen et al. (1995) Nucleic Acids Res 23:2661-2668; Hirschbein et al. (1997) Antisense Nucleic Acid Drug Dev 7:55-61). Merely to illustrate, the backbone of an dsRNA or siRNA can be modified with phosphorothioates, phosphoramidate, phosphodithioates, chimeric methylphosphonate-phosphodiesters, peptide nucleic acids, 5-propynyl-pyrimidine containing oligomers or sugar modifications (e.g., 2′-substituted ribonucleosides, a-configuration), in certain cases, the dsRNAs of the application lack 2′-hydroxy (2′-OH) containing nucleotides. In certain embodiments, the siRNA molecules comprise a phosphorothioate sense strand. In certain embodiments, the siRNA molecules comprise a phosphodiester antisense strand.

In a specific embodiment, at least one strand of the siRNA molecules has a 3′ overhang from about 1 to about 10 nucleotides in length, about 1 to 5 nucleotides in length, about 1 to 3 nucleotides in length, or about 2 to 4 nucleotides in length. In certain embodiments, an siRNA may comprise one strand having a 3′ overhang and the other strand is blunt-ended at the 3 end (e.g., does not have a 3′ overhang). In another embodiment, an siRNA may comprise a 3′ overhang on both strands. The length of the overhangs may be the same or different for each strand. In order to further enhance the stability of the siRNA, the 3′ overhangs can be stabilized against degradation. In one embodiment, the RNA is stabilized by including purine nucleotides, such as adenosine or guanosine nucleotides. Alternatively, substitution of pyrimidine nucleotides by modified analogues, e.g., substitution of uridine nucleotide 3′ overhangs by 2′-deoxythyinidine is tolerated and does not affect the efficiency of RNAi. The absence of a 2′ hydroxyl significantly enhances the nuclease resistance of the overhang in tissue culture medium and may be beneficial in vivo.

In another specific embodiment, the subject dsRNA can also be in the form of a long double-stranded RNA. For example, the dsRNA is at least 25, 50, 100, 200, 300 or 400 bases. In some cases, the dsRNA is 400-800 bases in length. Optionally, the dsRNAs are digested intracellularly, e.g., to produce siRNA sequences in the cell. However, use of long double-stranded RNAs in vivo is not always practical, presumably because of deleterious effects which may be caused by the sequence-independent dsRNA response. In such embodiments, the use of local delivery systems and/or agents which reduce the effects of interferon or PKR are preferred.

In a further specific embodiment, the dsRNA or siRNA is in the form of a hairpin structure (or hairpin RNA). The hairpin RNAs can be synthesized exogenously or can be formed by transcribing from RNA polymerase III promoters in vivo. Examples of making and using such hairpin RNAs for gene silencing in mammalian cells are described in, for example, Paddison et al., Genes Dev, 2002, 16:948-58; McCaffrey et al., Nature, 2002, 418:38-9; McManus et al., RNA, 2002, 8:842-50; Yu et al., Proc Natl Acad Sci USA, 2002, 99:6047-52. Preferably, such hairpin RNAs are engineered in cells or in an animal to ensure continuous and stable suppression of a target gene. It is known in the art that siRNAs can be produced by processing a hairpin RNA in the cell.

In one embodiment of this aspect of the invention, the ACSL4 inhibitor is administered in combination with a chemotherapeutic agent. In an exemplary embodiment the chemotherapeutic agent is docetaxel, capecitabine, cyclophosphamide and doxorubicin.

It will be readily apparent to one of ordinary skill in the relevant arts that other suitable modifications and adaptations to the methods and applications described herein can be made without departing from the scope of any of the embodiments. The following examples are included herewith for purposes of illustration only and are not intended to be limiting.

### EXAMPLES

### Example 1

### ACSL4 mRNA Expression as a Function of Estrogen Receptor Status in Tumor Samples

### Analysis of Gene Expression Arrays

Studies have been previously carried out to determine differences in gene expression that contribute to the hormone independent breast cancer phenotype. In several of these studies, gene expression arrays were utilized to catalog these differences. These studies produced, in addition to the gene expression array data for the specific proteins being studied, an enormous amount of gene expression array data which was not analyzed.

In an effort to identify differences in lipogenesis between normal and malignant tissue, a search was performed on the ONCOMINE database (Rhodes et al. 2004. Neoplasia, 6:1-6), a cancer microarray database and web-based data-mining platform. The search of the ONCOMINE cancer microarray database resulted in the identification of 10 separate studies (Miller et al., Proc. Natl. Acad. Sci. USA 102(38):12550-12555 (2005); Ivshina et al., Cancer Res. 66(21):10292-10301 (2006); van de Vijver et al., N. Engl. Med. 347(25); 1999-2009 (2002); Wang et al., Lancet 365(9460):671-679 (2005); Minn et al., Nature 436(7050):518-524 (2005); Sotiriou et al., J. Natl Cancer Inst. 98(4):262-272 (2006); Yu et al., Clin. Cancer Res. 12(11):3288-3296 (2006); Chin et al., Cancer Cell 10(6): 529-541 (2006); Saal et al., Proc Natl Acad Sci USA 104(18′0564-7569 (2007); Bittner et al., Natl. Center for Biotech. Infor., http://www.ncbi.nlm.nig.gov/geo/ (2005)) that included gene expression array data for ACSL4 mRNA, which was not analyzed,

### Results

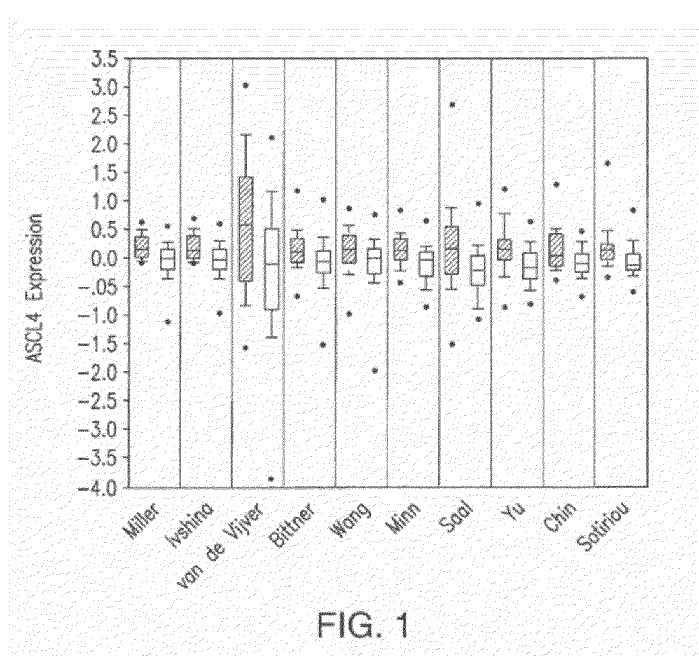

The analysis of the 10 separate studies showed overexpression of ACSL4 mRNA in human ER-negative breast tumor samples compared to ER-positive human breast tumor samples. FIG. 1 illustrates the results of ACSL4 mRNA expression as a function of estrogen receptor status in tumor samples from each of the 10 studies. Box-and-whisker plots indicate median, lower, and upper quartiles, and the smallest and largest values. In each study, ACSL4 mRNA gene expression data was higher in estrogen receptor negative samples, as shown by the gray shaded bars compared to the unshaded bars. The differences between the ER-positive and ER-negative values are significant with P<0.001 for all studies.

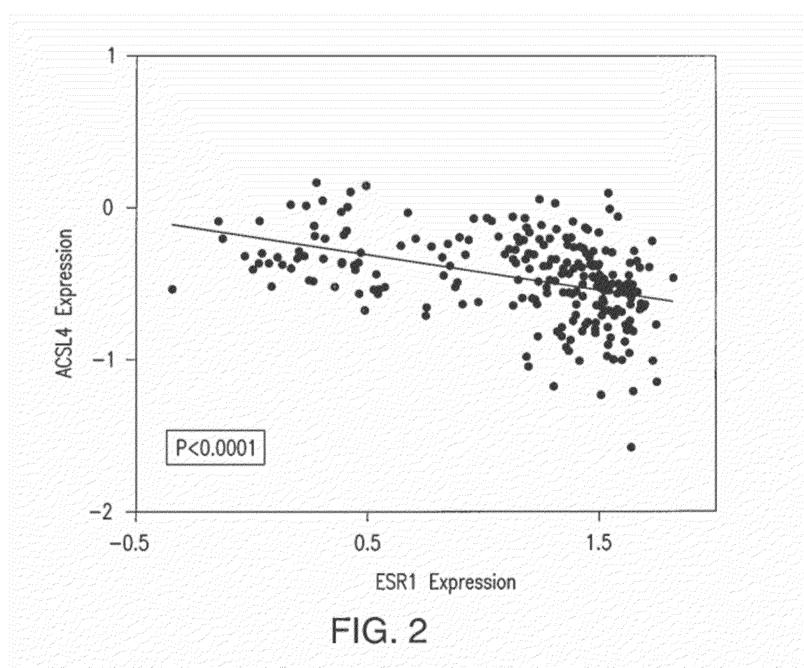

An analysis of the relationship between ACSL4 gene expression and ER gene expression was performed on the gene expression data provided in Miller el at, Proc. Natl. Acad. Sci. USA 102(38):12550-12555. The samples evaluated included 34 ER-negative and 213 ER-positive tumors. The relationship between the level of ACSL4 expression and ESR1 (ER) expression is illustrated in FIG. 2. The results show that there is a highly significant inverse correlation between expression levels of ER and ACSL4 mRNA (P<0.0001).

### Example 2

### Analysis of the Expression of ACSL4, ACSL3, ACSL4, ACSL5 and ACSL6 in ER-Positive and ER Negative Breast Cancer Cells Lines

### Analysis of Microarray Expression Data

An analysis was performed on the microarray expression data (Neve et al., Cancer Cell, 10(6):515-527 (2006), which is hereby incorporated by reference in its entirety) to determine the expression of the five ACSL isoforms (1, 3, 4, 5 and 6) in 50 human breast cancer cell lines as a function of ER status. A further analysis was performed on the 50 human breast cancer cell lines to determine the relationship between ER status and ACSL4 expression,

### Results

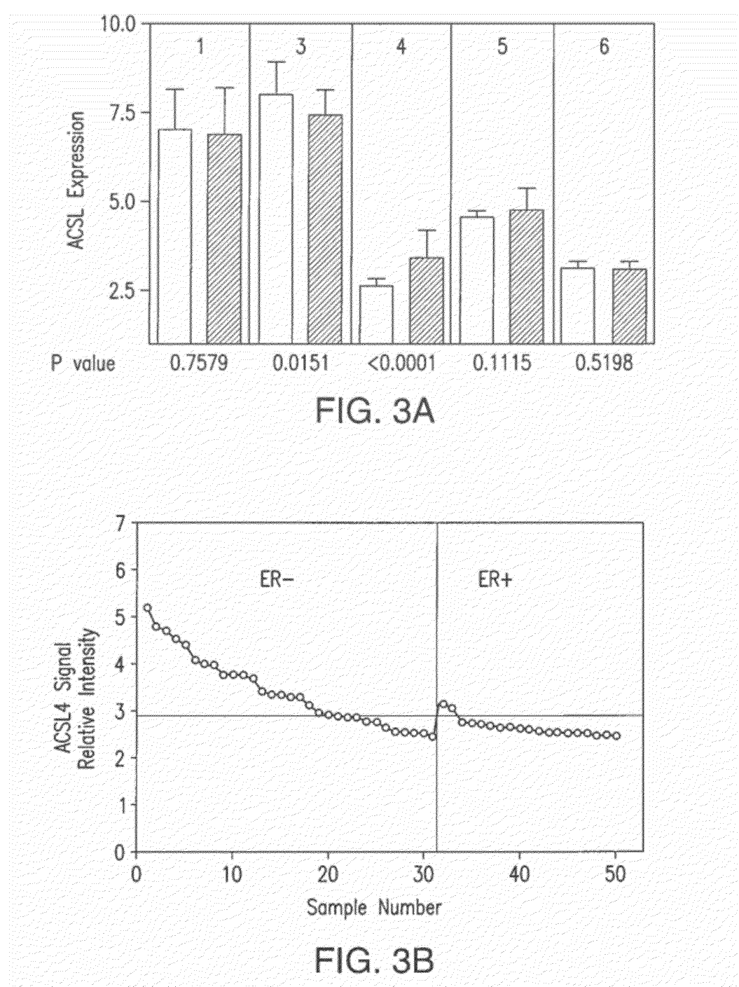

As illustrated in FIG. 3A, ACSL4 mRNA expression was significantly higher in ER-negative cells (P<0.0001), whereas expression of ACSL3 mRNA was significantly lower (P<0.015). There were no detectable differences in expression of ACSL1, 5, or 6 as a function of ER status. FIG. 3A shows the expression data for 19 ER-positive (unshaded bars) and 31 ER-negative (shaded bars) breast cancer cell lines for the five known isoforms of ACSL. The values provided are the mean±SD.

Of the 50 human breast cancer cell lines analyzed, 89% (17/19) of ER-positive cell lines were negative for ACSL4 mRNA expression and 65% (20/31) of ER-negative cell lines expressed ACSL4 mRNA, FIG. 3B illustrates the range of ACSL4 mRNA expression seen in the various cell lines. The horizontal line indicates the cutoff, as determined by immunoblot positivity.

### Example 3

### Analysis of ACSL4 Protein Expression in ER-Positive, ER-Negative, and AR-Positive and AR-Negative Breast Cancer Cells Lines

### Materials and Methods

ER-positive (MCF-7, MDA-MB-415 and T47D), ER-negative (BT20, MDA MB-231, and SKBR3), AR-positive (LNCaP and LNCaP-AD, and AR-negative (PC3 and DU145) cells were grown in either 96-well or 24-well plates at 37° C. in a humidified atmosphere in Dulbecco's minimal essential medium (high-glucose) containing Earle's salts and supplemented with 10% fetal bovine serum and antibiotics (penicillin [100 U/ml], Fungizone [0.25 μg/ml], and streptomycin [100 μg/ml]). All cell culture reagents were from Invitrogen (Carlsbad, Calif.).

After the cells in the 96-well or 24-well plates were washed with phosphate-buffered saline without calcium or magnesium, either 40 μl (96-well) or 200 μl (24-well) of sample buffer (10 mM Tris-HCl, 1 mM EDTA, 2.5% SDS, 5% β-mercaptoethanol, 0.01% bromophenol blue, pH 8.0) was added to the well. Samples were then heated to 95° C. for 5 minutes.

Electrophoresis was performed on either 1 or 4 μl of individual samples using the PhastGel System from GE Healthcare (Piscataway, N.J.). Precast 7.5% acrylamide gels were used with SDS buffer strips. Precision Plus protein standards from Bio-Rad (Hercules, Calif.) were used as molecular weight markers.

After separation, the proteins were transferred to a polyvinylidene fluoride membrane (Hydrabond-P) using the PhastGel transfer apparatus. The membrane was blocked with 5% milk in phosphate-buffered saline—Tween (0.1%) for 1 hour, followed by an overnight incubation with a 1:2000 dilution of affinity purified rabbit anti-ACSL4 antibody. A 1:5000 dilution of goat anti-rabbit HRP secondary antibody was used for the final step.

Signals were visualized using ECL-Plus chemilluminescence reagent. All immunoblot reagents were from GE Healthcare, with the exception of the antibody to β-actin, which was purchased from Cell Signaling Technologies (Danvers, Mass.). Quantitation of band densities was accomplished using the Quantity One program from Bio-Rad.

Relative differences in cell number were quantitated using the Cell Titer 96 AQᵤₑₒᵤₛ Reagent purchased from Promega (Madison, Wis.). Protocols used were as described by the manufacturer.

### Results

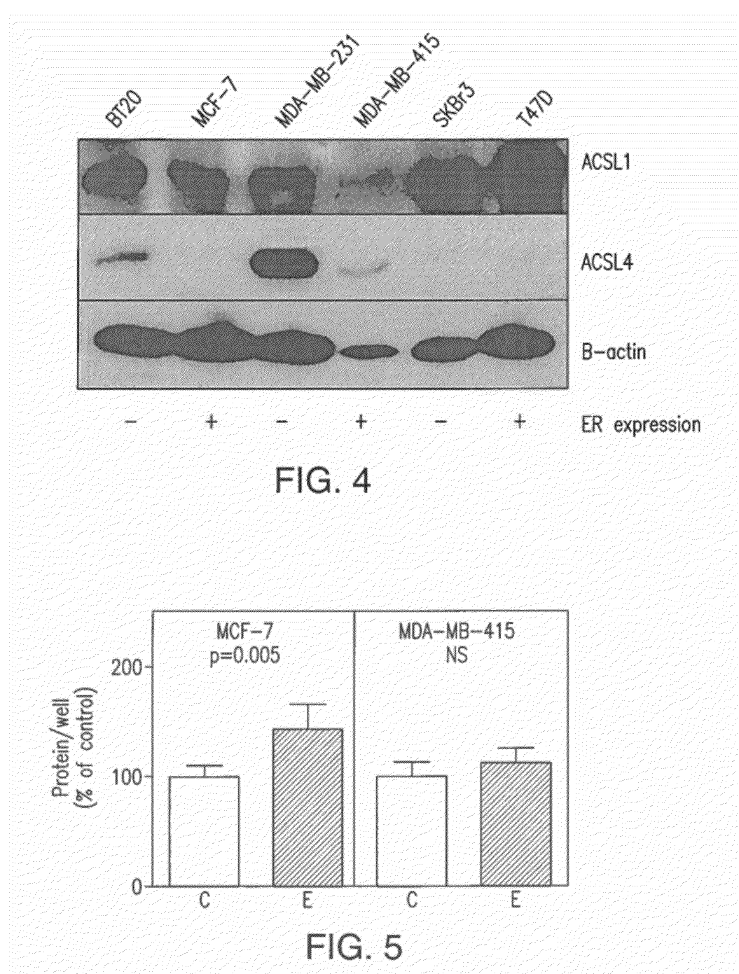

Cells were evaluated to determine whether the observed differences in ACSL4 mRNA expression shown in Examples 1 and 2 above were recapitulated at the protein level and whether these putative differences were an exclusive property of ACSL4. Accordingly, the levels of ACSL1 and ACSL4 protein relative to those of β-actin were assessed using immunoblot analyses of protein extracts isolated from ER-positive and ER-negative breast cancer cell lines. As illustrated in FIG. 4, all cell lines expressed detectable levels of ACSL1, which was consistent with microarray studies that demonstrated expression of ACSL1 mRNA in these cells as shown in FIG. 3A, FIG. 4 father illustrates that there was no correlation between ER status and ACSL1 protein expression levels.

With respect to ACSL4, as illustrated in FIG. 4, only those cells with normalized ACSL4 mRNA expression values greater than 2.9 (a predetermined threshold level) appeared positive for ACSL4 by immunoblot analysis. ACSL4 mRNA expression values for the cell lines tested are shown in Table 1.

TABLE 1
ER expression and ACSL4 mRNA expression values
for the breast cancer cell lines

| | | ACSL4 Expression |
|---|---|---|
| | | Value (Relative |
| | | Intensity to β-actin) - |
| | | Predetermined |
| Cell Line | ER Expression +/− | Threshold Level |
| MCF-7 | + | 2.52 |
| MDA-MB-415 | + | 3.14 |
| T47D | + | 2.62 |
| BT20 | − | 4.55 |
| MDA-MB-231 | − | 5.20 |
| SKBR3 | − | 2.77 |

### Example 4

### Analysis of the Growth Response of Estrogen Receptor Positive and ACSL4 Receptor Positive Cells Lines to Estrogen

As indicated above in the results of Example 2, two cell lines which were ER-positive where also ACSL4-positive. A study comparing cell growth in the presence of estrogen of one of these cell lines (MDA-MB-415) to a cell line which is ER-positive and ACSL4 negative (MCF-7) was performed.

Additionally, a study was performed in which cell growth in the presence of estrogen of the estrogen dependent MCF-7 cells, which are ACSL4 negative, were compared to modified MCF-7 cells, which were induced to express ACSL4.

### Results

MDA-MB-415 cells, as illustrated in FIG. 5, showed no significant growth response to estrogen (E) compared to the control (C) cell line. In contrast, MCF-7 cells, which were ACSL4-negative showed a significant growth response (p=0.005) compared to the control cells. Accordingly, ACSL4 expression is indicative of estrogen independent growth even in the presence of the estrogen receptor.

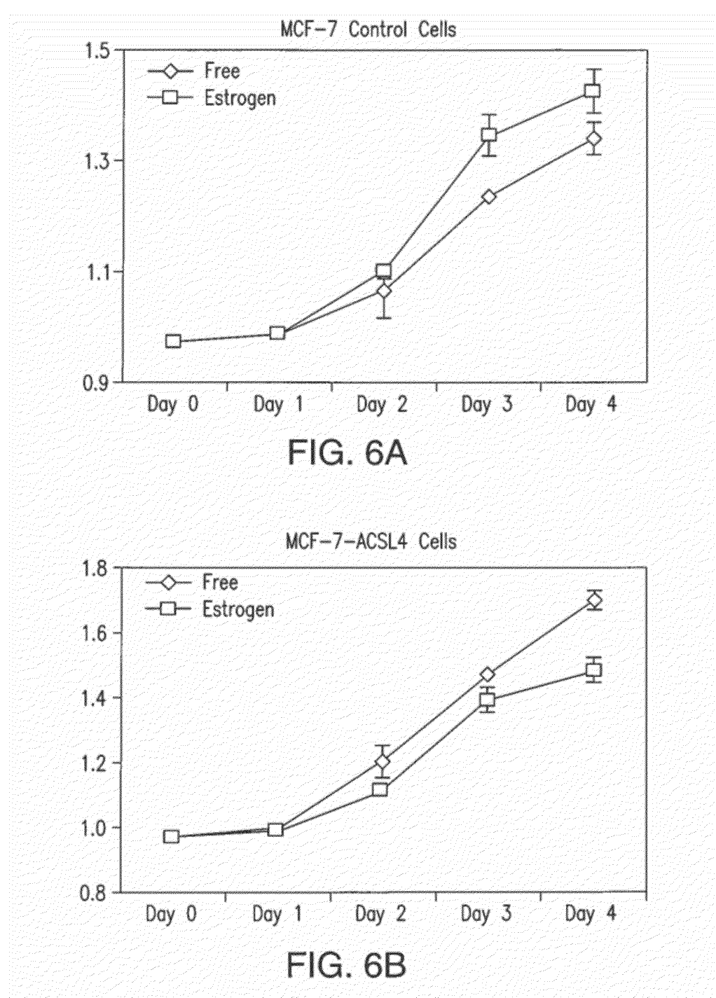

MCF-7 control cells, which are ACSL4-negative, had a proliferative growth effect in the presence of estrogen compared to estrogen-free conditions, as shown in FIG. 6A. In contrast, as shown in FIG. 6B, MCF-7 cells genetically manipulated to express ACSL4 became unresponsive to the proliferative effect of estrogen and actually grew less when in the presence of estrogen.

### Example 5

### Effect of ACSL4 Ablation on MDA-MB-231 Cells

Since ACSL4 experimental data appears to suggest that ACSL4 expression is associated with sex steroid hormone independent growth, a study was performed to determine whether ablation of ACSL4 enzymatic activity would impact the ability of cells to proliferate. Clearly, in certain instances, ACSL4 activity was not required for proliferation as evidenced by the ability of cells lacking ACSL4, such as MCF-7 and T47D cells, to proliferate.

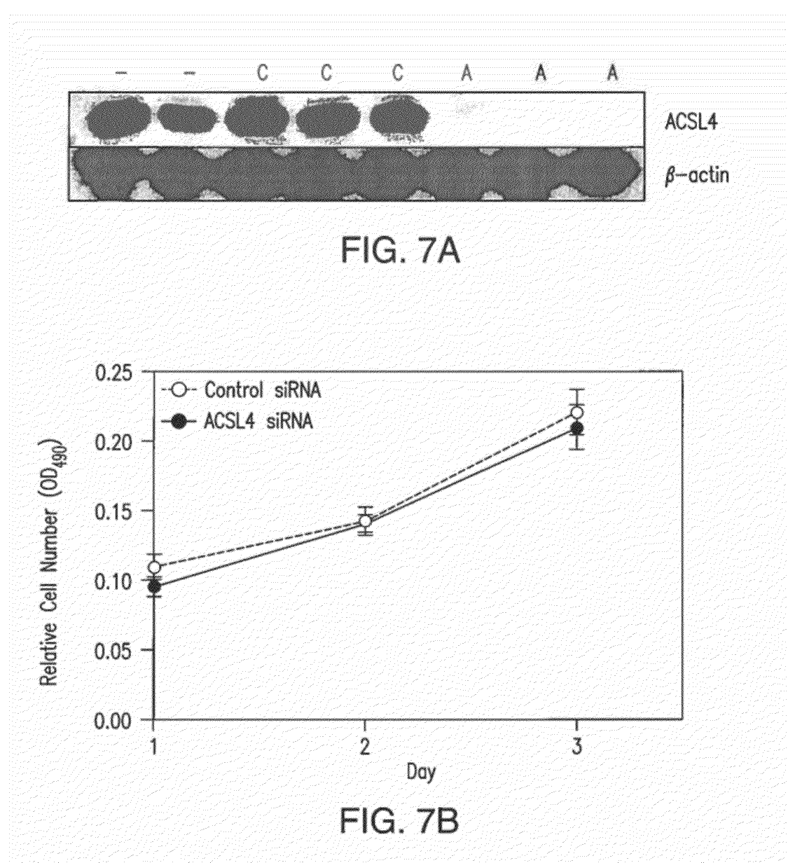

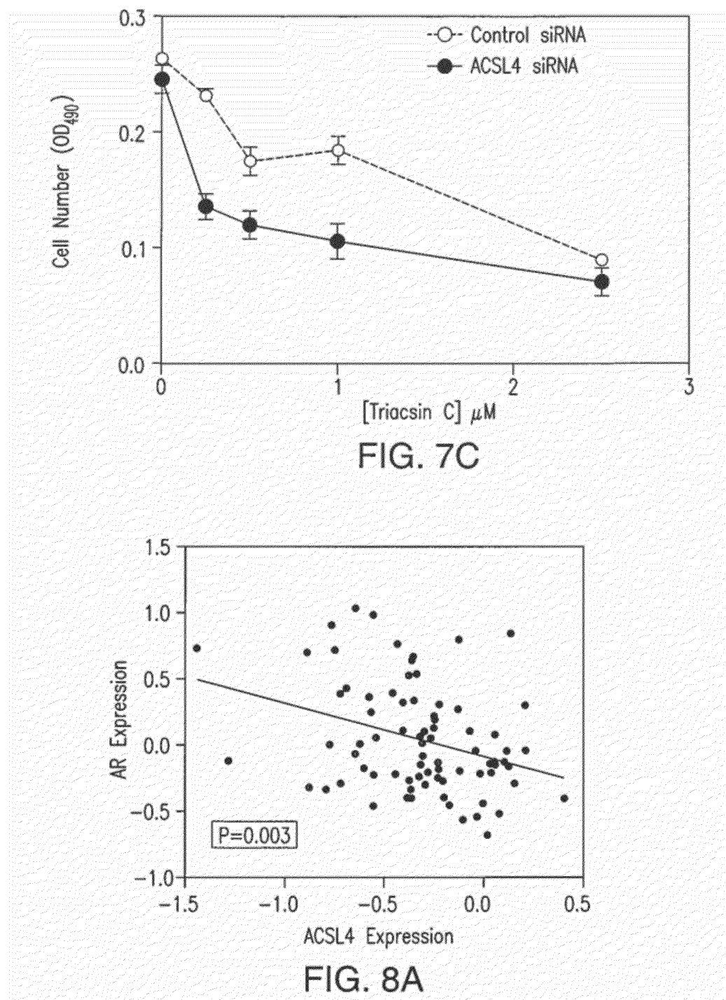

MDA-MB-231 cells were treated for 48 hours with either a control or ACSL4-specific siRNA. The siRNA was a SmartPool siRNA obtained from Dharmacon, consisting of 4 separate siRNAs. As shown in FIG. 7A, a greater than 95% reduction in ACSL4 protein expression was achieved by treating the cells with the ACSL4-specific siRNA. Moreover, the knockdown effect persisted for at least three days after removal of the transfection medium. When proliferation of the known-down cells was compared with that of control cells, no difference was observed as demonstrated in FIG. 7B.

Next, the effect of triacsin C treatment on MDA-MB-231 control and knockdown cells were compared. Triacsin C is specific for inhibition of ACSL1, 3 and 4, with little or no effect on the activities of ACSL5 or 6, Van Horn et al., Biochemistry 44(5):1635-1642 (2005). The half-maximal inhibitory concentration values reported for ACSL1, 3 and 4 indicate that ACSL1 is the most sensitive, whereas ACS14 is the least sensitive. In addition, this reagent has been demonstrated to inhibit proliferation and induce apoptosis in a variety of cancer cells. Mashima et al., Cancer Sci., 100: 1556-1562 (2009).

The results shown in FIG. 7C indicate that ablation of cells ACSL4 resulted in a three-fold increase in triacsin C sensitivity. The half-maximal inhibitory concentration for triacsin C was 1.59 μM for control cells and 0.56 μM for ACSL4 knockdown cells. These results suggest that AMA activity makes a significant contribution to the overall ACSL activity required for growth and survival of the MDA-MB-231

### Example 6

### Analysis of the Expression of ACSL4 in Androgen Receptor Positive Breast Cancer Cells Lines

A subset of mammary tumors known as molecular apocrine are ER-negative and AR-positive. An analysis of microarray expression data in a subset of breast cancers and in basal (ER-negative, AR-negative) and luminal (ER-positive, AR-Positive) breast tumors (Farmer et al., Oncogene, 24(29):4660-4671, (2005)), showed that ACSL4 mRNA levels were significantly lower in the molecular apocrine samples compared with the basal subset (P<0.001). Interestingly, of the 11 ER-negative cell lines from Example 2 that do not express ACSL4, three showed high levels of expression of AR mRNA. One of these cell lines, MDA-MB-453, has been shown to have a positive proliferative response to androgens. Doane et al., Oncogene, 25(28):3994-4008, (2006).

To assess whether the expression of ACSL4 and AR were inversely related, results from microarray studies (Wang et al., Lancet, 9460:671-679, (2005)) in an ER negative subset of tumors were evaluated. AR and ACSL4 expression levels in prostate cancer cell lines were also compared, Microarray data from a study that assessed mRNA expression in prostate cancer cells (Zhao et al., Prostate, 63(2):187-197, (2005)) was analyzed and determined that ACSL4 mRNA was over-expressed in AR-negative cell lines.

In order to determine whether any observed differences in mRNA were recapitulated at the protein level, ACSL4 and β-actin expression in extracts from two AR-negative cell lines (PC2 and DU145), one AR-positive cell line (LNCaP), and one AR-positive cell line that is androgen-independent for growth (LNCaP-AI) were analyzed. Additionally, results from an mRNA expression study in human prostate tumors (Holzbeierlein et al. Am. J. Pathol., 164(1):217-227, (2004)) were analyzed.

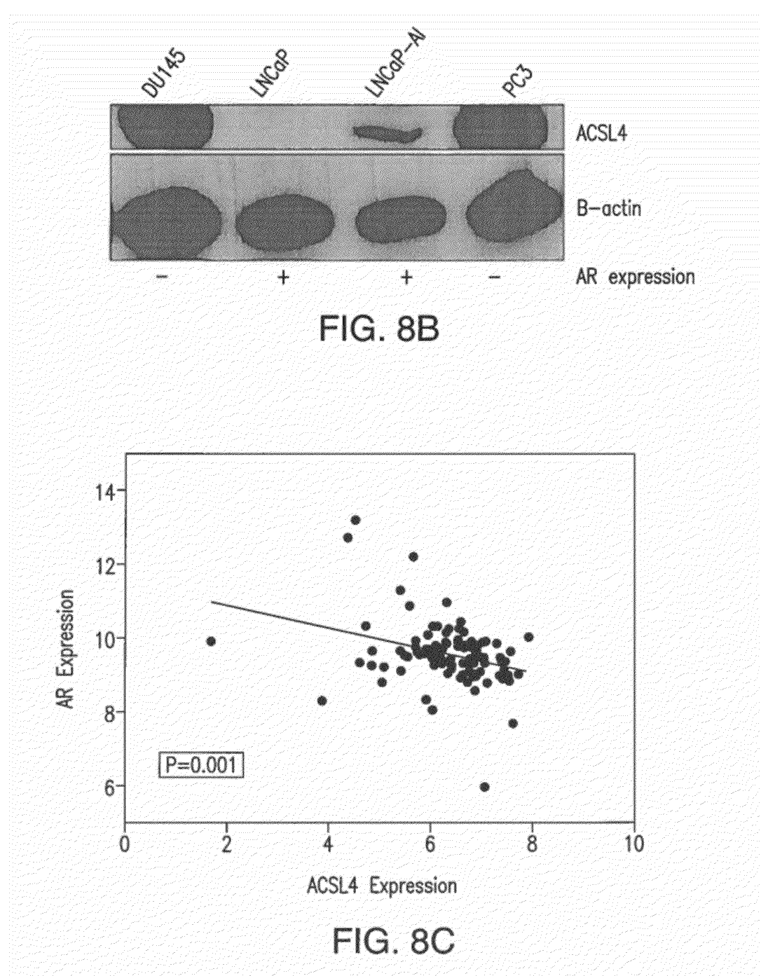

Interestingly, as shown in FIG. 8A, the results of the analysis of the microarray studies from Wang et al showed a significant inverse correlation between AR and ACSL4 mRNA expression. Additionally, as shown in FIG. 8B, both of the AR-negative cell lines expressed high levels of ACSL4, whereas the AR-positive line did not express the protein. Importantly, the results shown in FIG. 8B indicate that a loss of androgen sensitivity in LNCaPAI cell was associated with increased expression of ACSL4, even in the presence of AR. Furthermore, the analysis of the results from the mRNA expression study by Holzheierlein et al. found that ACSL4 were inversely correlated with AR expression as indicated in FIG. 8C. The combined results of this example strongly suggest negative correlation between the AR receptor and ACSL4 expression that could reflect functional relationships in growth requirements or signaling events involving these proteins.

### Example 7

### Analysis of the Expression of ACSL4 in HER2/neu Receptor Positive Breast Cancer Cells Lines

### Analysis of Microarray Expression Data

An analysis was performed on the microarray expression data for 50 human breast cancer cell lines (Neve et. al., Cancer Cell, 10(6):515-527 (2006), which is hereby incorporated by reference in its entirety) to determine the relationship between HER2 expression and ACSL4 expression.

### Results

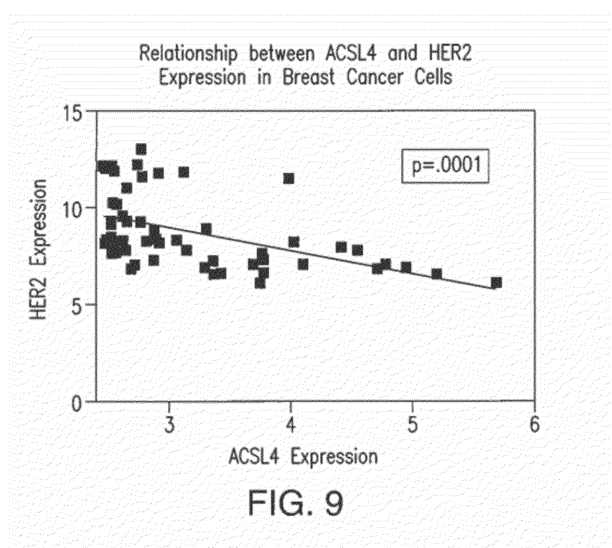

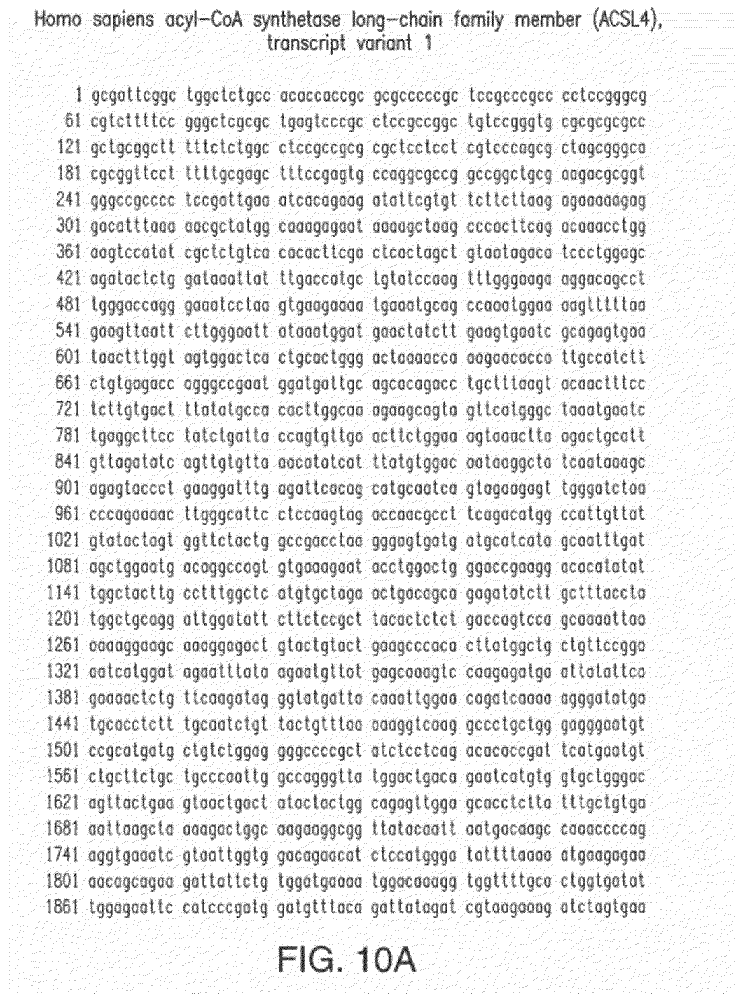

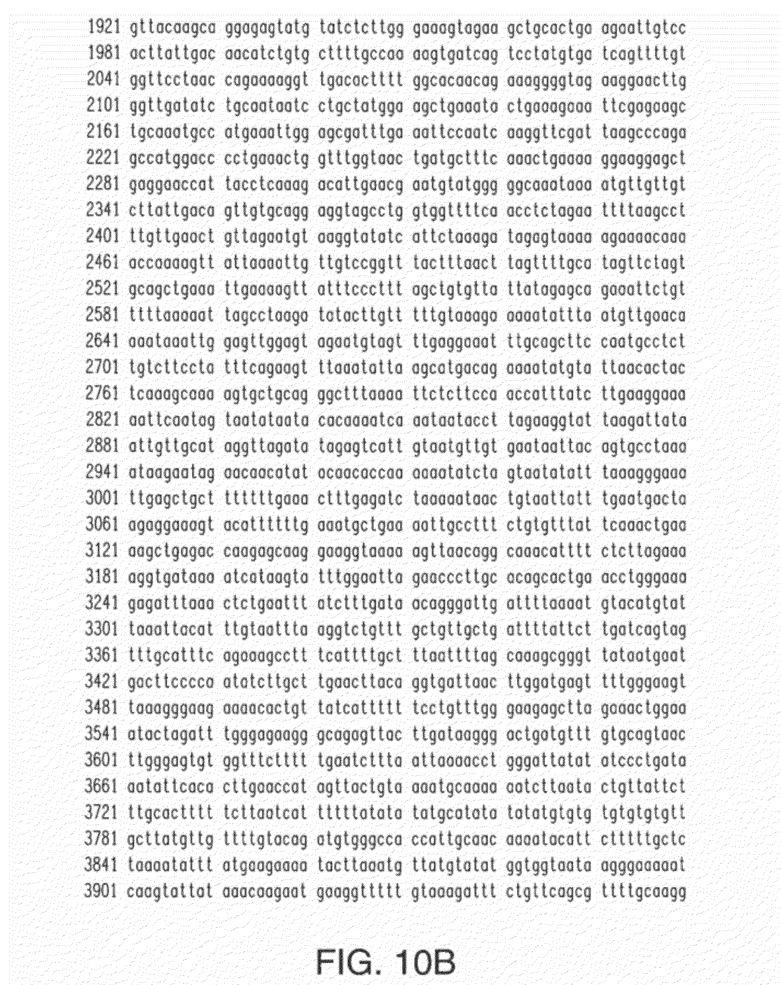

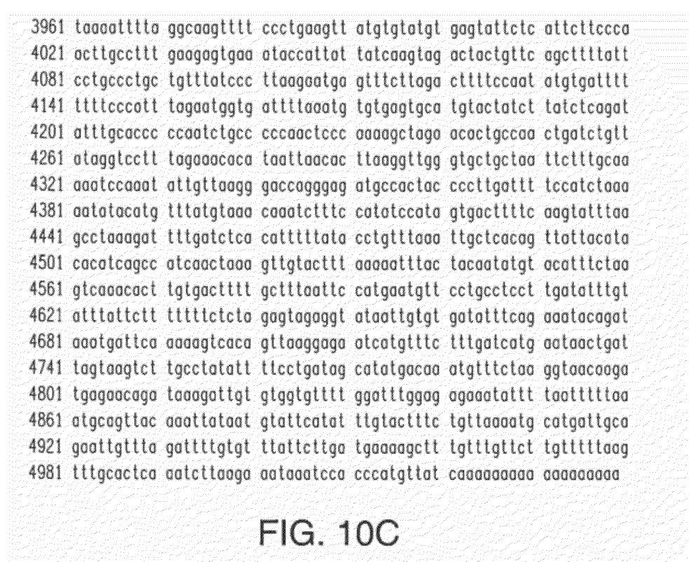

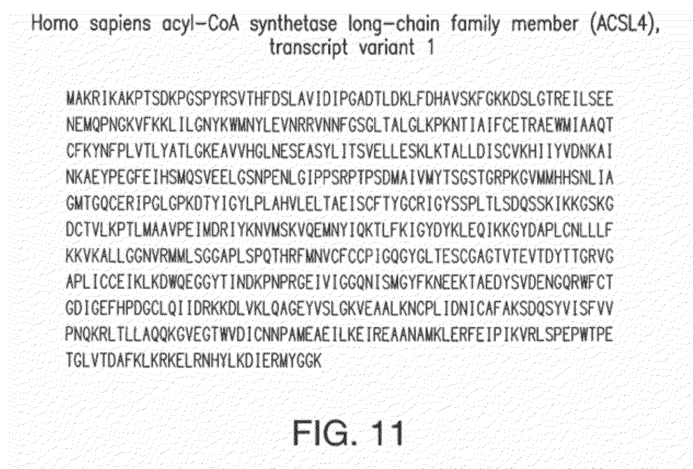

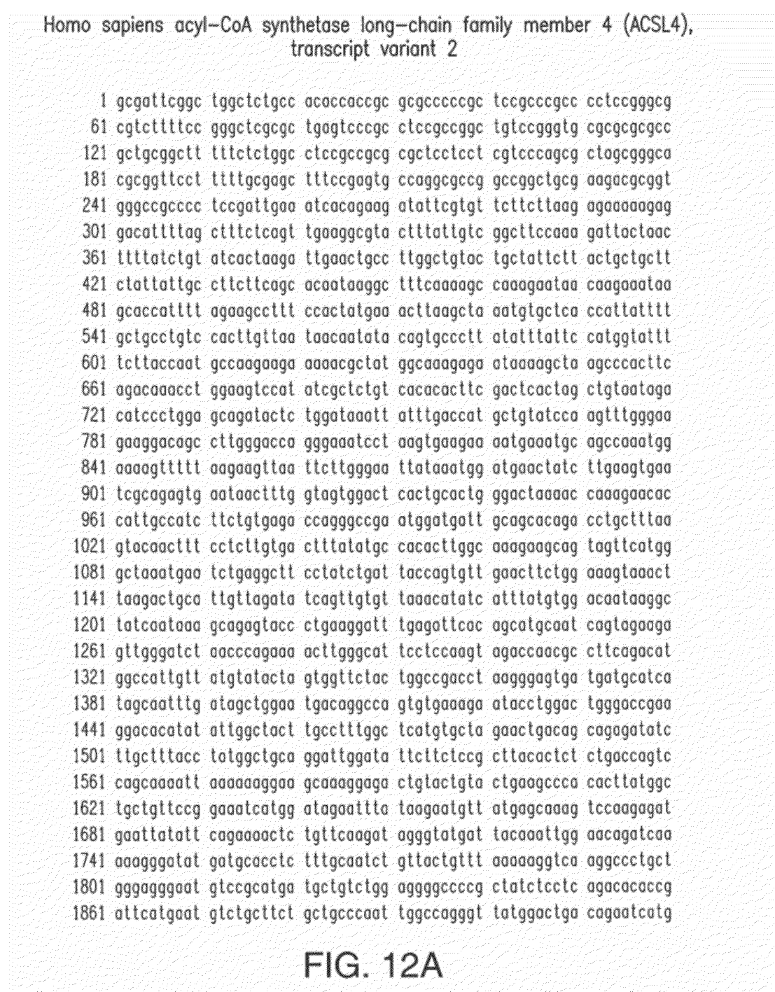

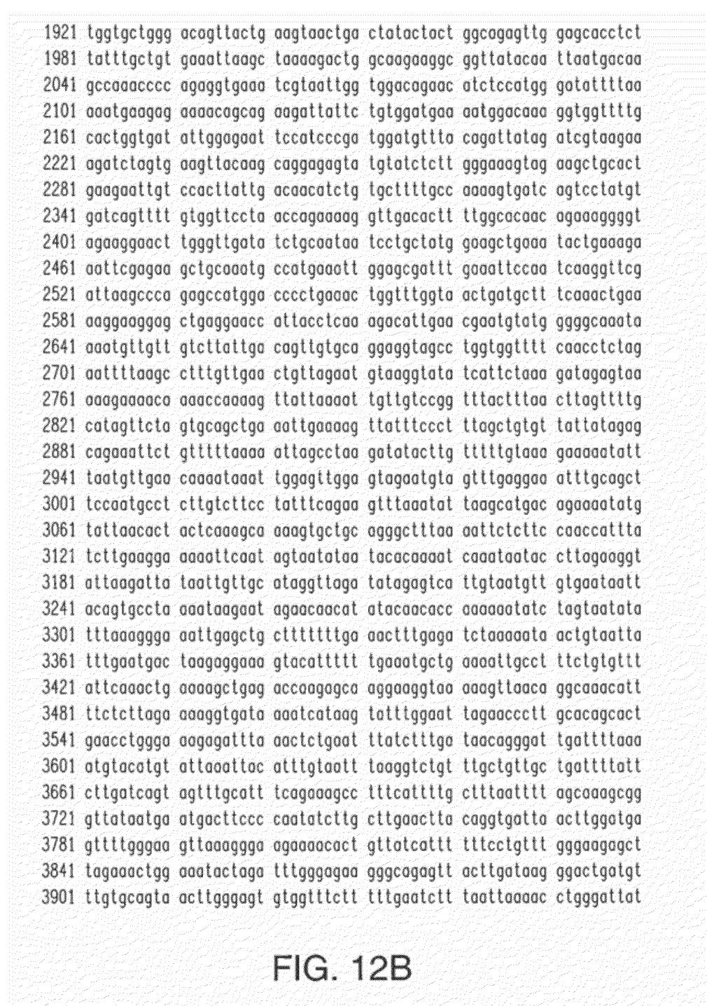

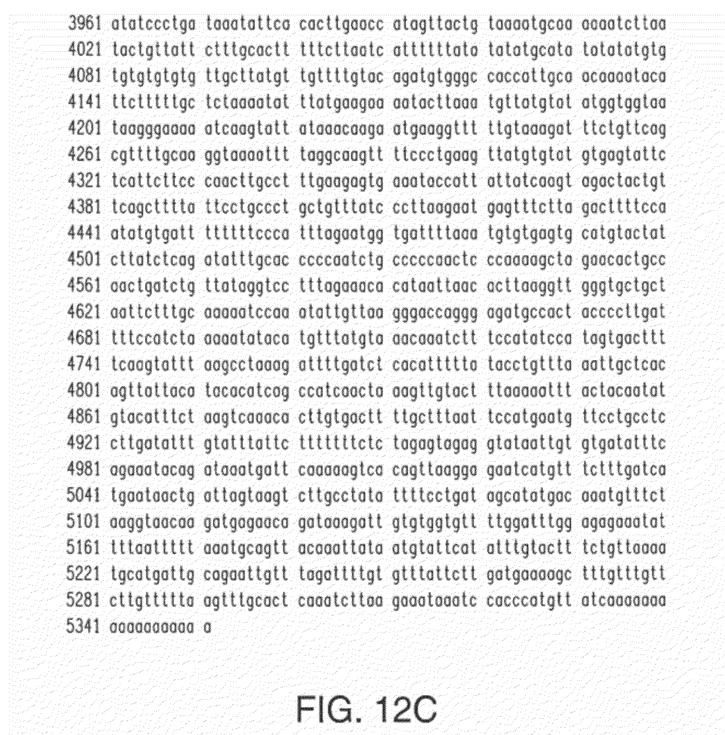

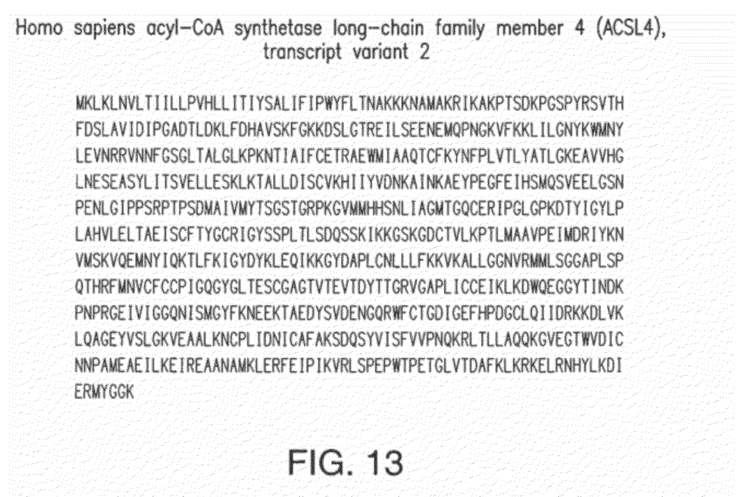

The results shown in FIG. 9 indicate that expression of ACSL4 is negatively correlated with expression of HER2. Consequently, human breast cancer cell lines which expressed ACSL4 were ER-negative as well as HER2-negative, in contrast, SKBR3 while ER-negative, express high levels of HER2 as determined from public databases and literature searches.

### Example 8

### Analysis of the Hormone/Growth Factor Receptor Status and ACSL4 Expression in Seventy-Three Breast Cancer Cells Lines

Two separate studies were performed to determine the relationship between ACSL4 expression and that of human epidermal growth factor receptor 2 (HER2), estrogen receptor (ER), progesterone receptor (PR) and androgen receptor (AR) in 73 different breast cancer cells lines. (Neve et, al., Cancer Cell, 10(6):515-527 (2006) and Hoeflich et al., Clinical Cancer Research, 15:4649-4664 (2009)).

TABLE 2
ACSL4 Expression in Hormone/Growth Factor Receptor
Positive Breast Cancer Cell Lines

| Cell Line | ACSL4 | HER2 | ER | PR | AR |
|---|---|---|---|---|---|
| 600MPE | N | N | P | N | N |
| AU565 | N | P | N | N | N |
| BT474 | N | P | P | P | P |
| BT483 | N | N | P | N | N |
| Cama1 | N | N | P | N | P |
| EFM19 | N | N | P | N | N |
| EFM192A | N | P | P | N | N |
| HCC1007 | N | P | P | N | P |
| HCC1008 | N | N | P | N | N |
| HCC1419 | N | P | P | N | N |
| HCC1428 | N | N | P | N | N |
| HCC1500 | N | N | P | N | P |
| HCC1569 | P | P | N | N | N |
| HCC1954 | P | P | N | N | N |
| HCC202 | N | P | N | N | P |
| HCC2218 | N | P | N | N | P |
| KPL1 | N | N | P | N | N |
| KPL4 | N | P | P | N | P |
| LY2 | N | N | P | N | N |
| MCF7 | N | N | P | P | N |
| MDA-134VI | N | N | P | N | P |
| MDA175VII | N | N | P | N | N |
| MDA361 | N | N | P | P | N |
| MDA415 | P | N | P | N | P |
| MDA453 | N | N | N | N | P |
| MDA468 | N | N | N | N | N |
| MFM223 | N | N | N | N | P |
| SKBr3 | N | P | N | N | N |
| SUM185PE | N | N | N | N | P |
| SUM190PT | P | P | N | N | N |
| SUM225CWN | N | P | N | N | N |
| SUM44PE | N | N | P | N | P |
| SUM52PE | N | N | P | N | N |
| T47D | N | N | P | P | N |
| UACC812 | N | P | P | N | N |
| UACC893 | N | P | N | N | P |
| ZR75-1 | N | N | P | N | P |
| ZR75-30 | N | P | P | N | P |
| ZR75B | P | N | P | N | N |

TABLE 3
ACSL4 Expression in Hormone/Growth Factor Receptor
Negative Breast Cancer Cell Lines

| Cell Line | ACSL4 | HER2 | ER | PR | AR |
|---|---|---|---|---|---|
| BT-20 | P | N | N | N | N |
| ET549 | P | N | N | N | N |
| CAL120 | P | N | N | N | N |
| CAL148 | N | N | N | N | N |
| CAL51 | N | N | N | N | N |
| CAL85-1 | P | N | N | N | N |
| DU4475 | P | N | N | N | N |
| EVSA-T | N | N | N | N | N |
| HCC1143 | N | N | N | N | N |
| HCC1187 | N | N | N | N | N |
| HCC1395 | P | N | N | N | N |
| HCC1500 | N | N | N | N | N |
| HCC1599 | P | N | N | N | N |
| HCC1806 | N | N | N | N | N |
| HCC1937 | P | N | N | N | N |
| HCC2157 | P | N | N | N | N |
| HCC2185 | N | N | N | N | N |
| HCC3153 | P | N | N | N | N |
| HCC38 | P | N | N | N | N |
| HCC70 | P | N | N | N | N |
| HDQ-P1 | P | N | N | N | N |
| HS578T | P | N | N | N | N |
| JIMT1 | P | N | N | N | N |
| MCF10A | P | N | N | N | N |
| MCF12A | P | N | N | N | N |
| MDA231 | P | N | N | N | N |
| MDA435s | P | N | N | N | N |
| MDA436 | P | N | N | N | N |
| MDAMB157 | P | N | N | N | N |
| MX1 | P | N | N | N | N |
| SUM1315 | P | N | N | N | N |
| SUM149PT | P | N | N | N | N |
| SUM159PT | P | N | N | N | N |
| SW527 | P | N | N | N | N |

Tables 2 and 3 illustrate the relationship between ACSL4 expression and hormone/growth factor receptor status. The data confirm the inverse relationship between ACSL4 expression and ER, PR, HER2, and AR receptor expression in breast cancer cell lines and tumor samples, as supported by mRNA expression data (cell lines and tumors) as wells as immunoblot analysis. Monaco et al. Transl. Oncology, 3:91-98 (2010). As shown in Table 1, only 118% (5139) of cells lines which are ER, PR, HER2 and/or AR receptor positive express ACSL4. In contrast, as shown in Table 2, 76.5% (26134) of ER, PR, HER2 and/or AR receptor negative cell lines express ACSL4.

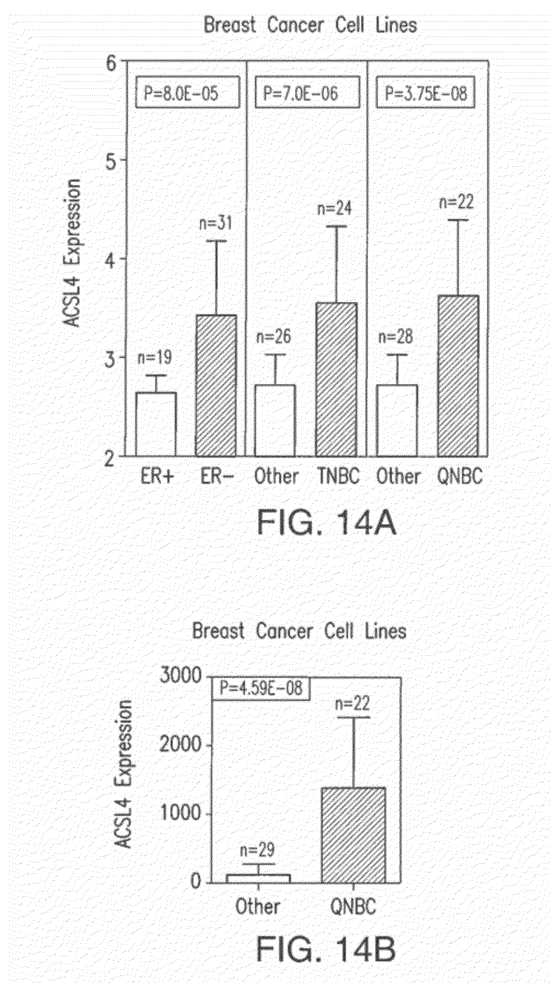

As shown in FIG. 14, when the data from the hormone/growth factor receptor breast cancer positive and negative cell lines are expressed separately, the significance of the difference in ACSL4 expression levels between receptor-positive and receptor-negative cell lines is similar in both studies (p value=3-5E⁻⁰⁸). As shown in FIG. 14, the significance of the difference in ACSL4 expression increased when comparing only ER− cells to ER+, to comparing triple negative breast cancer cells lines (ER−, PR−, and HER2−) to cells which are not triple negative, and finally to comparing quadruple negative breast cancer cell lines (QNBC) (ER−, PR−, HER2−, and AR−) to cells which are not quadruple negative,

TABLE 4
Differential expression of ACSL4 in triple negative tumor samples.

| Study Author | Fold Change | P Value |
|---|---|---|
| Waddell | 5.6 | 4.2E−07 |
| Richardson Breast 2 | 1.8 | 0.002 |
| TGCA Breast | 1.5 | 7.3E−05 |

Fold change is the ratio of values for triple negative samples versus samples expressing one or more receptor (ER, PR, HER2). Data from www.oncomine.com

As shown in Table 4, when the data from three separate studies were evaluated, expression of ACSL4 mRNA was significantly higher in TNBC samples than in samples that expressed one or more of the receptor biomarkers (ER, PR, HER2). The values for the androgen receptor (AR) were not available so the comparison is between the triple negative breast cancer cell lines and the others (i.e., ER, PR, HER2) as reported by the Oracomine database (www.oncomine.com). In three separate studies, expression of ACSL4 mRNA was significantly higher in TNBC than in samples that expressed one or more of the receptor biomarkers. The highest and most significant difference was seen in the study by Waddell, which is likely attributed to the use of laser capture microdissection to extract RNA from the samples resulting in decreased contamination with other cell types. Waddell at al., Breast Cancer Research and Treatment, 123:661-677 (2010). Both stromal cells and the majority of normal mammary epithelium cells were positive for ACSL4, which was not surprising since neither stromal cells nor the majority of luminal cells express ER. Anderson at al., J. of Mammary Gland Biology and Neoplasia, 9:3-13 (2004).

### Example 9

### Analysis of the Co-Expression of Hormone/Growth Factor Receptors and ACSL4 on Resistance to Hormone-Based Targeted Therapies

### Estrogen Based Therapy

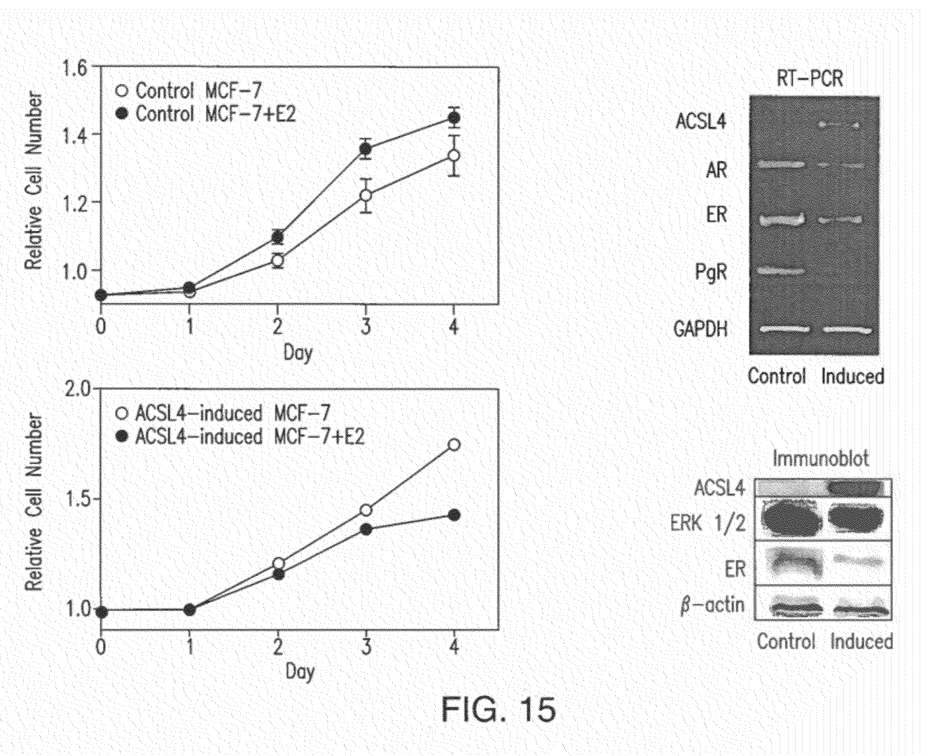

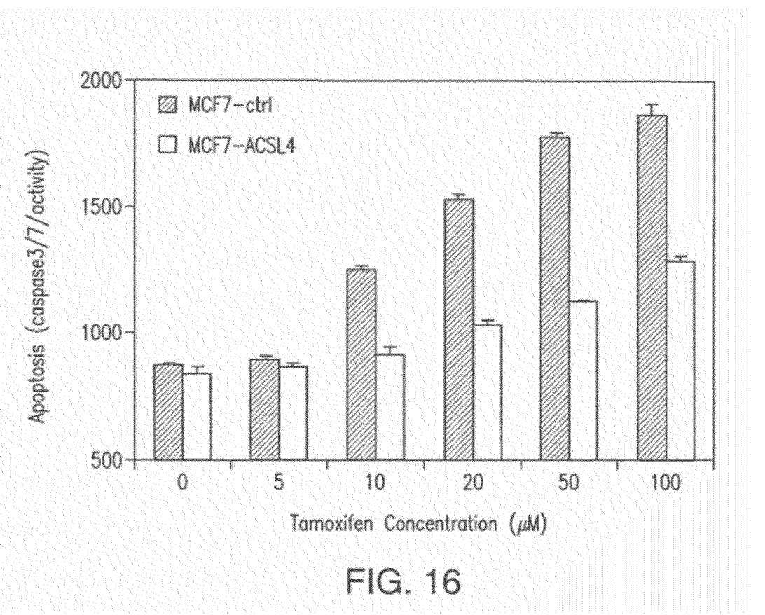

An analysis was performed on breast cancer cell lines that are hormone receptor positive and positive for ACSL4 expression. As shown in FIG. 5, when estrogen was added to one such breast cancer cell line, MDA-MB-415, there was no increase in cellular proliferation. In the present study, estrogen responsive MCF-7 cells, which are ACSL4-negative, were forced to express AMA. The forced expression resulted in a loss of responsiveness to estrogen, accompanied by a decrease in expression of ER, PR and AR (FIG. 15) and a decrease in the sensitivity to tamoxifen treatment (FIG. 16). The results were confirmed using conditional and stable transfection techniques.

### HER2 Based Therapy

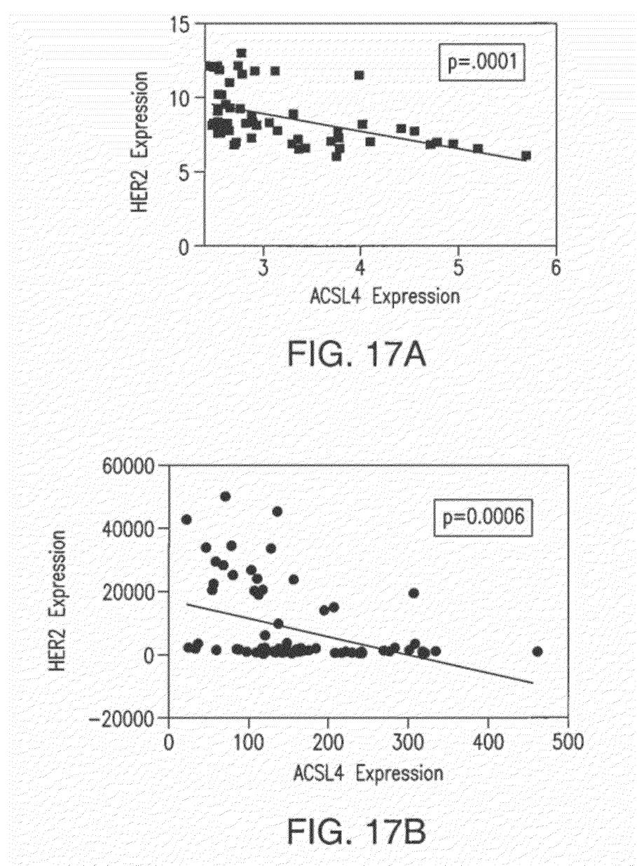

As Table 2 indicates, the breast cancer cell lines HCC1569, HCC1954 and SUM190PT simultaneously express ACSL4 and HER2. In most instances, as shown in FIG. 17, there is a negative correlation between HER2 and ACSL4 expression in either cell lines or tumor samples. Thus, simultaneous expression would render cells resistant to HER based therapies even in the presence of excess HER2.

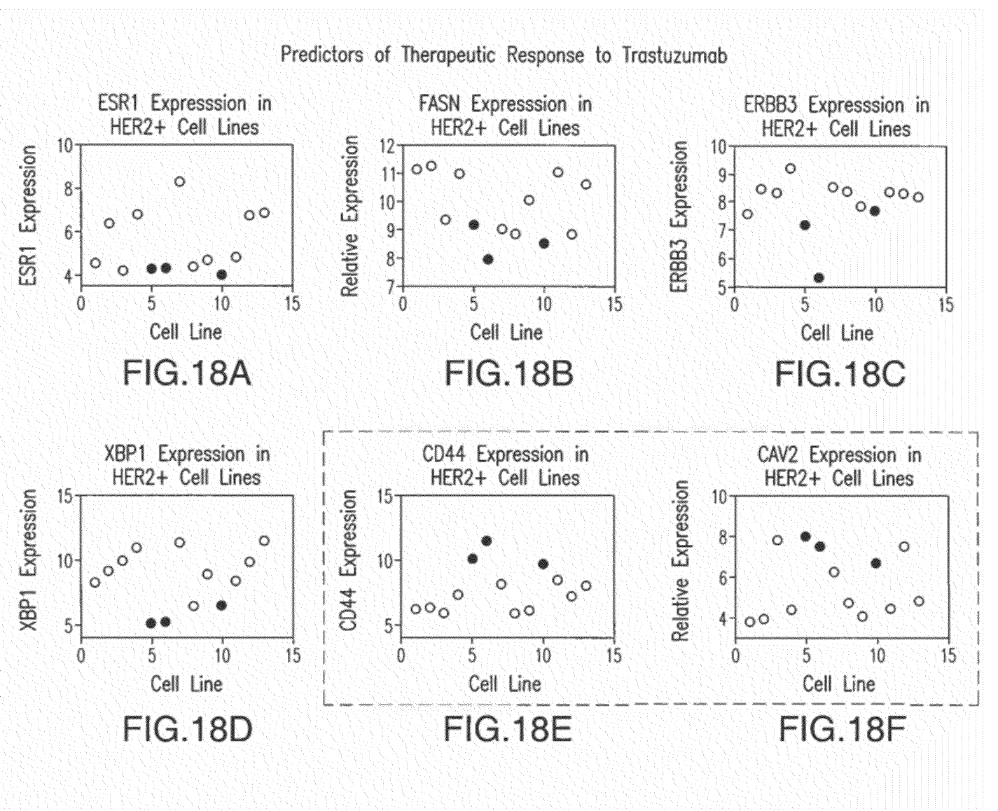

A review of the expression patterns of proteins which are either positively or negatively associated with breast cancer cell line response to trastuzumab, a HER2-based therapy was conducted. The expression patterns of these proteins can be used to predict responsiveness to trastuzumab. FIG. 18, illustrates expression patterns with respect to six putative makers of trastuzumab sensitivity for eleven HER2+ breast cancer cell lines, including the three previously noted cells lines which express both HER and ACSL4. Filled circles represent cell lines that are positive for both ACSL4 and HER2. Empty circles are HER 2+ and ACSL4-negative. Interestingly, the three cell lines which express ACSL4 have relatively low levels of expression of proteins that correlate positively with response to trastuzumab (Panels A-D), whereas the three ACSL4+_cell lines have high levels of expression of proteins which are associated with resistance to trastuzumab, (Panels E-F).

What is claimed is:

1. A method for predicting the responsiveness of a patient to a cancer treatment regimen, said method comprising:
a. obtaining a biological sample from the patient;
b. assaying the biological sample for expression of long-chain fatty acyl-CoA synthetase 4 (ACSL4);
c. quantitating the level of ACSL4 expression in the biological sample;
d. comparing the level of ACSL4 expression in the biological sample to the level of ACSL4 expression in a control sample or comparing the level of ACSL4 expression in the biological sample to a threshold; and
c. determining that the patient is responsive to a cancer treatment regimen where the level of ACSL4 expression in the biological sample is less than the level of ACSL4 expression in the control sample or less than the threshold for determining that the patient is not responsive to a cancer treatment regimen where the level of ACSL4 expression in the biological sample is greater than the level of ACSL4 expression in the control sample or greater than the predetermined threshold level.

2. The method of claim 1, wherein the biological sample is a tumor biopsy.

3. The method of claim 2, wherein the tumor biopsy is a fine needle aspiration and cytology (FNAC), core biopsy or an excisional biopsy.

4. The method of claim 1, wherein the step of assaying is performed by immunohistochemistry (WIC) or western blot.

5. The method of claim 4, wherein the immunohistochemistry uses a detectably labeled antibody specific for the ACSL4.

6. The method of claim 5, wherein the antibody is a detectably labeled monoclonal antibody.

7. The method of claim 5, wherein the label is a chromagen or fluorophore.

8. The method of claim 1, wherein the step of assaying is performed by detection of ACSL4 mRNA from the biological sample using reverse-transcriptase polymerase chain reaction (RT-PCR).

9. The method of claim 1, wherein the cancer treatment regimen is a breast cancer treatment.

10. The method of claim 1, wherein the breast cancer treatment regimen is a hormonal-based therapy.

11. The method of claim 10, wherein the hormonal-based therapy is a selective estrogen receptor modulator (SERM), selective estrogen down-regulator (SERD) or an aromatase inhibitor.

12. The method of claim 11, wherein the SERM is tamoxifen, raloxifene, toremifene, or lasofoxifene.

13. The method of claim 9, wherein the breast cancer treatment regimen is a receptor tyrosine kinase inhibitor.

14. The method of claim 13, wherein the receptor tyrosine kinase inhibitor is an antibody.

15. The method of claim 14, wherein the antibody is a monoclonal antibody.

16. The method of claim 15, wherein the monoclonal antibody is directed against HER-2/neu or EGFR.

17. The method of claim 15, wherein the monoclonal antibody is cetuximab (Erbitux), panitumumab, zalutumumab, nimotuzumab, matuzumab, or trastuzumab (Herceptin).

18. The method of claim 13, wherein the receptor tyrosine kinase inhibitor is a small molecule inhibitor.

19. The method of claim 18, wherein the small molecule inhibitor is gefitinib, erlotinib or lapatinib.

20. The method of claim 1, wherein the control sample is obtained from the patient from which the biological sample was obtained.

21. The method of claim 1, wherein the control sample is obtained from a different patient from which the biological sample was obtained.

22. The method of claim 1, wherein the control sample is non-cancerous cells or tissue.

23. The method of claim 1, wherein the predetermined threshold level is set as a maximum amount of ACSL4 in a biological sample in which a patient is responsive to treatment with a cancer treatment regimen.

24. A method for identifying whether a breast cancers expresses estrogen receptor (ER), progesterone receptor (PR), human epidermal growth factor 2 (HER2/neu) and/or androgen receptor (AR), said method comprising:
a obtaining a biological sample from the patient;
b, assaying the biological sample for expression of ACSL4;
c. quantitating the level of ACSL4 expression in the biological sample;
d. comparing the level of ACSL4 expression in the biological sample to the level of ACSL4 expression in a control sample or comparing the level of ACSL4 expression in the biological sample to a threshold; and
e. determining that the patient is negative for the expression of estrogen receptor (ER), progesterone receptor (PR), human epidermal growth factor 2 (HER2/neu) and/or androgen receptor (AR) where the level of ACSL4 expression in the biological sample is greater than the level of ACSL4 expression in the control sample or greater than the predetermined threshold level or determining that the patient is positive for the expression of estrogen receptor (ER), progesterone receptor (PR), human epidermal growth factor 2 (HER2/neu) and/or androgen receptor (AR) where the level of ACSL4 expression in the biological sample is less than the level of ACSL4 expression in the control sample or less than the predetermined threshold level.

25. A method for determining a breast cancer adjuvant treatment regimen for a cancer patient, said method comprising:
a. obtaining a biological sample from the patient;
b. assaying the biological sample for expression of ACSL4
c. quantitating the level of ACSL4 expression in the biological sample;
d. comparing the level of ACSL4 expression in the biological sample to the level of ACSL4 expression in a control sample or comparing the level of ACSL4 expression in the biological sample to a threshold; and
e. determining a breast cancer adjuvant treatment where the level of ACSL4 expression in the biological sample is less than the level of ACSL4 expression in the control sample or less than the predetermined threshold level.

26. The method of claim 25, wherein the biological sample is a tumor biopsy.

27. The method of claim 26, wherein the tumor biopsy is a fine needle aspiration and cytology (FNAC), core biopsy or an excisional biopsy.

28. The method of claim 25, wherein the step of assaying is performed by immunohistochemistry (IHC) or western blot.

29. The method of claim 28, wherein the immunohistochemistry uses a detectably labeled antibody specific for the ACSL4.

30. The method of claim 29, wherein the antibody is a detectably labeled monoclonal antibody.

31. The method of claim 29, wherein the label is a chromagen or fluorophore.

32. The method of claim 25, wherein the step of assaying is performed by detection of ACSL4 mRNA from the biological sample using reverse transcriptase polymerase chain reaction (RT-PCR).

33. The method of claim 25, wherein the breast cancer adjuvant treatment regimen is a hormonal-based therapy.

34. The method of claim 33, wherein the hormonal-based therapy is a selective estrogen receptor modulator (SERM), selective estrogen down-regulator (SERD) or an aromatase inhibitor.

35. The method of claim 34, wherein the SERM is tamoxifen, raloxifene, toremifene, or lasofoxifene.

36. The method of claim 25, wherein the breast cancer adjuvant treatment regimen is a receptor tyrosine kinase inhibitor.

37. The method of claim 36, wherein the receptor tyrosine kinase inhibitor is an antibody.

38. The method of claim 37, wherein the antibody is a monoclonal antibody.

39. The method of claim 38, wherein the monoclonal antibody is directed against HER 2/neu or EGFR.

40. The method of claim 38, wherein the monoclonal antibody is cetuximab (Erbitux), panitumumab, zalutumumab, nimotuzumab, matuzumab, or trastuzumab (Herceptin).

41. The method of claim 36, wherein the receptor tyrosine kinase inhibitor is a small molecule inhibitor.

42. The method of claim 41, wherein the small molecule inhibitor is gefitinib, eriotinib or lapatinib.

43. The method of claim 25, wherein the control sample is obtained from the patient from which the biological sample was obtained.

44. The method of claim 25, Wherein the control sample is obtained from a different patient from which the biological sample was obtained.

45. The method of claim 25, wherein the control sample: is non-cancerous cells or tissue.

46. The method of claim 25, wherein the predetermined threshold level is set as a maximum amount of ACSL4 in a biological sample in which a patient is responsive to treatment with a cancer treatment regimen.

47. A method of treating a patient having breast cancer comprising administering a breast cancer therapeutic regimen comprising an ACSL4 inhibitor.

48. The method of claim 47 wherein the ACSL4 inhibitor is a small molecule.

49. The method of claim 47 wherein the ACSL4 inhibitor is a nucleic acid inhibitor.

50. The method of claim 49 wherein the nucleic acid inhibitor is an siRNA, dsRNA, miRNA, antisense RNA, aptamer, ribozyme, or an enzymatic nucleic acid.

51. The method of claim 47 wherein the breast cancer therapeutic regimen further comprises a chemotherapeutic agent.

52. The method of claim 51 wherein the chemotherapeutic agent is anthracycline, taxane, cyclophosphamide, capecitabine, vinorelbine, or gemcitabine.


SEQUENCE LISTING (4 sequences)

SEQ ID NO: 1 - DNA, 5039 bp, Homo sapiens
gcgattcggctggctctgccacaccaccgcgcgcccccgctccgcccgcccctccgggcgcgtcttttccgggctcgcgctgagtcccgcctccgccggctgtccgggtgcgcgcgcgccgctgcggctttttctctggcctccgccgcgcgctcctcctcgtcccagcgctagcgggcacgcggttcctttttgcgagctttccgagtgccaggcgccggccggctgcgaagacgcggtgggccgcccctccgattgaaatcacagaagatattcgtgttcttcttaagagaaaaagaggacatttaaaaacgctatggcaaagagaataaaagctaagcccacttcagacaaacctggaagtccatatcgctctgtcacacacttcgactcactagctgtaatagacatccctggagcagatactctggataaattatttgaccatgctgtatccaagtttgggaagaaggacagccttgggaccagggaaatcctaagtgaagaaaatgaaatgcagccaaatggaaaagtttttaagaagttaattcttgggaattataaatggatgaactatcttgaagtgaatcgcagagtgaataactttggtagtggactcactgcactgggactaaaaccaaagaacaccattgccatcttctgtgagaccagggccgaatggatgattgcagcacagacctgctttaagtacaactttcctcttgtgactttatatgccacacttggcaaagaagcagtagttcatgggctaaatgaatctgaggcttcctatctgattaccagtgttgaacttctggaaagtaaacttaagactgcattgttagatatcagttgtgttaaacatatcatttatgtggacaataaggctatcaataaagcagagtaccctgaaggatttgagattcacagcatgcaatcagtagaagagttgggatctaacccagaaaacttgggcattcctccaagtagaccaacgccttcagacatggccattgttatgtatactagtggttctactggccgacctaagggagtgatgatgcatcatagcaatttgatagctggaatgacaggccagtgtgaaagaatacctggactgggaccgaaggacacatatattggctacttgcctttggctcatgtgctagaactgacagcagagatatcttgctttacctatggctgcaggattggatattcttctccgcttacactctctgaccagtccagcaaaattaaaaaaggaagcaaaggagactgtactgtactgaagcccacacttatggctgctgttccggaaatcatggatagaatttataagaatgttatgagcaaagtccaagagatgaattatattcagaaaactctgttcaagatagggtatgattacaaattggaacagatcaaaaagggatatgatgcacctctttgcaatctgttactgtttaaaaaggtcaaggccctgctgggagggaatgtccgcatgatgctgtctggaggggccccgctatctcctcagacacaccgattcatgaatgtctgcttctgctgcccaattggccagggttatggactgacagaatcatgtggtgctgggacagttactgaagtaactgactatactactggcagagttggagcacctcttatttgctgtgaaattaagctaaaagactggcaagaaggcggttatacaattaatgacaagccaaaccccagaggtgaaatcgtaattggtggacagaacatctccatgggatattttaaaaatgaagagaaaacagcagaagattattctgtggatgaaaatggacaaaggtggttttgcactggtgatattggagaattccatcccgatggatgtttacagattatagatcgtaagaaagatctagtgaagttacaagcaggagagtatgtatctcttgggaaagtagaagctgcactgaagaattgtccacttattgacaacatctgtgcttttgccaaaagtgatcagtcctatgtgatcagttttgtggttcctaaccagaaaaggttgacacttttggcacaacagaaaggggtagaaggaacttgggttgatatctgcaataatcctgctatggaagctgaaatactgaaagaaattcgagaagctgcaaatgccatgaaattggagcgatttgaaattccaatcaaggttcgattaagcccagagccatggacccctgaaactggtttggtaactgatgctttcaaactgaaaaggaaggagctgaggaaccattacctcaaagacattgaacgaatgtatgggggcaaataaaatgttgttgtcttattgacagttgtgcaggaggtagcctggtggttttcaacctctagaattttaagcctttgttgaactgttagaatgtaaggtatatcattctaaagatagagtaaaaagaaaacaaaaccaaaagttattaaaattgttgtccggtttactttaacttagttttgcatagttctagtgcagctgaaattgaaaagttatttccctttagctgtgttattatagagcagaaattctgtttttaaaaattagcctaagatatacttgtttttgtaaagaaaaatatttaatgttgaacaaaataaattggagttggagtagaatgtagtttgaggaaatttgcagcttccaatgcctcttgtcttcctatttcagaagtttaaatattaagcatgacagaaaatatgtattaacactactcaaagcaaaagtgctgcagggctttaaaattctcttccaaccatttatcttgaaggaaaaattcaatagtaatataatacacaaaatcaaataataccttagaaggtattaagattataattgttgcataggttagatatagagtcattgtaatgttgtgaataattacagtgcctaaaataagaatagaacaacatatacaacaccaaaaaatatctagtaatatatttaaagggaaattgagctgctttttttgaaactttgagatctaaaaataactgtaattatttgaatgactaagaggaaagtacattttttgaaatgctgaaaattgcctttctgtgtttattcaaactgaaaagctgagaccaagagcaaggaaggtaaaaagttaacaggcaaacattttctcttagaaaaggtgataaaatcataagtatttggaattagaacccttgcacagcactgaacctgggaaagagatttaaactctgaatttatctttgataacagggattgattttaaaatgtacatgtattaaattacatttgtaatttaaggtctgtttgctgttgctgattttattcttgatcagtagtttgcatttcagaaagcctttcattttgctttaattttagcaaagcgggttataatgaatgacttccccaatatcttgcttgaacttacaggtgattaacttggatgagttttgggaagttaaagggaagaaaacactgttatcattttttcctgtttgggaagagcttagaaactggaaatactagatttgggagaagggcagagttacttgataagggactgatgtttgtgcagtaacttgggagtgtggtttctttttgaatctttaattaaaacctgggattatatatccctgataaatattcacacttgaaccatagttactgtaaaatgcaaaaaatcttaatactgttattctttgcactttttcttaatcattttttatatatatgcatatatatatgtgtgtgtgtgtgttgcttatgttgttttgtacagatgtgggccaccattgcaacaaaatacattctttttgctctaaaatatttatgaagaaaatacttaaatgttatgtatatggtggtaataagggaaaaatcaagtattataaacaagaatgaaggtttttgtaaagatttctgttcagcgttttgcaaggtaaaattttaggcaagttttccctgaagttatgtgtatgtgagtattctcattcttcccaacttgcctttgaagagtgaaataccattattatcaagtagactactgttcagcttttattcctgccctgctgtttatcccttaagaatgagtttcttagacttttccaatatgtgattttttttcccatttagaatggtgattttaaatgtgtgagtgcatgtactatcttatctcagatatttgcacccccaatctgcccccaactcccaaaagctagaacactgccaactgatctgttataggtcctttagaaacacataattaacacttaaggttgggtgctgctaattctttgcaaaaatccaaatattgttaagggaccagggagatgccactaccccttgattttccatctaaaaatatacatgtttatgtaaacaaatctttccatatccatagtgacttttcaagtatttaagcctaaagattttgatctcacatttttatacctgtttaaattgctcacagttattacatacacatcagccatcaactaaagttgtactttaaaaatttactacaatatgtacatttctaagtcaaacacttgtgacttttgctttaattccatgaatgttcctgcctccttgatatttgtatttattctttttttctctagagtagaggtataattgtgtgatatttcagaaatacagataaatgattcaaaaagtcacagttaaggagaatcatgtttctttgatcatgaataactgattagtaagtcttgcctatattttcctgatagcatatgacaaatgtttctaaggtaacaagatgagaacagataaagattgtgtggtgttttggatttggagagaaatattttaatttttaaatgcagttacaaattataatgtattcatatttgtactttctgttaaaatgcatgattgcagaattgtttagattttgtgtttattcttgatgaaaagctttgtttgttcttgtttttaagtttgcactcaaatcttaagaaataaatccacccatgttatcaaaaaaaaaaaaaaaaaa

SEQ ID NO: 2 - protein, 670 aa, Homo sapiens
MAKRIKAKPTSDKPGSPYRSVTHFDSLAVIDIPGADTLDKLFDHAVSKFGKKDSLGTREILSEENEMQPNGKVFKKLILGNYKWMNYLEVNRRVNNFGSGLTALGLKPKNTIAIFCETRAEWMIAAQTCFKYNFPLVTLYATLGKEAVVHGLNESEASYLITSVELLESKLKTALLDISCVKHIIYVDNKAINKAEYPEGFEIHSMQSVEELGSNPENLGIPPSRPTPSDMAIVMYTSGSTGRPKGVMMHHSNLIAGMTGQCERIPGLGPKDTYIGYLPLAHVLELTAEISCFTYGCRIGYSSPLTLSDQSSKIKKGSKGDCTVLKPTLMAAVPEIMDRIYKNVMSKVQEMNYIQKTLFKIGYDYKLEQIKKGYDAPLCNLLLFKKVKALLGGNVRMMLSGGAPLSPQTHRFMNVCFCCPIGQGYGLTESCGAGTVTEVTDYTTGRVGAPLICCEIKLKDWQEGGYTINDKPNPRGEIVIGGQNISMGYFKNEEKTAEDYSVDENGQRWFCTGDIGEFHPDGCLQIIDRKKDLVKLQAGEYVSLGKVEAALKNCPLIDNICAFAKSDQSYVISFVVPNQKRLTLLAQQKGVEGTWVDICNNPAMEAEILKEIREAANAMKLERFEIPIKVRLSPEPWTPETGLVTDAFKLKRKELRNHYLKDIERMYGGK

SEQ ID NO: 3 - DNA, 5351 bp, Homo sapiens
gcgattcggctggctctgccacaccaccgcgcgcccccgctccgcccgcccctccgggcgcgtcttttccgggctcgcgctgagtcccgcctccgccggctgtccgggtgcgcgcgcgccgctgcggctttttctctggcctccgccgcgcgctcctcctcgtcccagcgctagcgggcacgcggttcctttttgcgagctttccgagtgccaggcgccggccggctgcgaagacgcggtgggccgcccctccgattgaaatcacagaagatattcgtgttcttcttaagagaaaaagaggacattttagctttctcagttgaaggcgtactttattgtcggcttccaaagattactaacttttatctgtatcactaagattgaactgccttggctgtactgctattcttactgctgcttctattattgccttcttcagcacaataaggctttcaaaagccaaagaataacaagaaataagcaccattttagaagcctttccactatgaaacttaagctaaatgtgctcaccattattttgctgcctgtccacttgttaataacaatatacagtgcccttatatttattccatggtattttcttaccaatgccaagaagaaaaacgctatggcaaagagaataaaagctaagcccacttcagacaaacctggaagtccatatcgctctgtcacacacttcgactcactagctgtaatagacatccctggagcagatactctggataaattatttgaccatgctgtatccaagtttgggaagaaggacagccttgggaccagggaaatcctaagtgaagaaaatgaaatgcagccaaatggaaaagtttttaagaagttaattcttgggaattataaatggatgaactatcttgaagtgaatcgcagagtgaataactttggtagtggactcactgcactgggactaaaaccaaagaacaccattgccatcttctgtgagaccagggccgaatggatgattgcagcacagacctgctttaagtacaactttcctcttgtgactttatatgccacacttggcaaagaagcagtagttcatgggctaaatgaatctgaggcttcctatctgattaccagtgttgaacttctggaaagtaaacttaagactgcattgttagatatcagttgtgttaaacatatcatttatgtggacaataaggctatcaataaagcagagtaccctgaaggatttgagattcacagcatgcaatcagtagaagagttgggatctaacccagaaaacttgggcattcctccaagtagaccaacgccttcagacatggccattgttatgtatactagtggttctactggccgacctaagggagtgatgatgcatcatagcaatttgatagctggaatgacaggccagtgtgaaagaatacctggactgggaccgaaggacacatatattggctacttgcctttggctcatgtgctagaactgacagcagagatatcttgctttacctatggctgcaggattggatattcttctccgcttacactctctgaccagtccagcaaaattaaaaaaggaagcaaaggagactgtactgtactgaagcccacacttatggctgctgttccggaaatcatggatagaatttataagaatgttatgagcaaagtccaagagatgaattatattcagaaaactctgttcaagatagggtatgattacaaattggaacagatcaaaaagggatatgatgcacctctttgcaatctgttactgtttaaaaaggtcaaggccctgctgggagggaatgtccgcatgatgctgtctggaggggccccgctatctcctcagacacaccgattcatgaatgtctgcttctgctgcccaattggccagggttatggactgacagaatcatgtggtgctgggacagttactgaagtaactgactatactactggcagagttggagcacctcttatttgctgtgaaattaagctaaaagactggcaagaaggcggttatacaattaatgacaagccaaaccccagaggtgaaatcgtaattggtggacagaacatctccatgggatattttaaaaatgaagagaaaacagcagaagattattctgtggatgaaaatggacaaaggtggttttgcactggtgatattggagaattccatcccgatggatgtttacagattatagatcgtaagaaagatctagtgaagttacaagcaggagagtatgtatctcttgggaaagtagaagctgcactgaagaattgtccacttattgacaacatctgtgcttttgccaaaagtgatcagtcctatgtgatcagttttgtggttcctaaccagaaaaggttgacacttttggcacaacagaaaggggtagaaggaacttgggttgatatctgcaataatcctgctatggaagctgaaatactgaaagaaattcgagaagctgcaaatgccatgaaattggagcgatttgaaattccaatcaaggttcgattaagcccagagccatggacccctgaaactggtttggtaactgatgctttcaaactgaaaaggaaggagctgaggaaccattacctcaaagacattgaacgaatgtatgggggcaaataaaatgttgttgtcttattgacagttgtgcaggaggtagcctggtggttttcaacctctagaattttaagcctttgttgaactgttagaatgtaaggtatatcattctaaagatagagtaaaaagaaaacaaaaccaaaagttattaaaattgttgtccggtttactttaacttagttttgcatagttctagtgcagctgaaattgaaaagttatttccctttagctgtgttattatagagcagaaattctgtttttaaaaattagcctaagatatacttgtttttgtaaagaaaaatatttaatgttgaacaaaataaattggagttggagtagaatgtagtttgaggaaatttgcagcttccaatgcctcttgtcttcctatttcagaagtttaaatattaagcatgacagaaaatatgtattaacactactcaaagcaaaagtgctgcagggctttaaaattctcttccaaccatttatcttgaaggaaaaattcaatagtaatataatacacaaaatcaaataataccttagaaggtattaagattataattgttgcataggttagatatagagtcattgtaatgttgtgaataattacagtgcctaaaataagaatagaacaacatatacaacaccaaaaaatatctagtaatatatttaaagggaaattgagctgctttttttgaaactttgagatctaaaaataactgtaattatttgaatgactaagaggaaagtacattttttgaaatgctgaaaattgcctttctgtgtttattcaaactgaaaagctgagaccaagagcaaggaaggtaaaaagttaacaggcaaacattttctcttagaaaaggtgataaaatcataagtatttggaattagaacccttgcacagcactgaacctgggaaagagatttaaactctgaatttatctttgataacagggattgattttaaaatgtacatgtattaaattacatttgtaatttaaggtctgtttgctgttgctgattttattcttgatcagtagtttgcatttcagaaagcctttcattttgctttaattttagcaaagcgggttataatgaatgacttccccaatatcttgcttgaacttacaggtgattaacttggatgagttttgggaagttaaagggaagaaaacactgttatcattttttcctgtttgggaagagcttagaaactggaaatactagatttgggagaagggcagagttacttgataagggactgatgtttgtgcagtaacttgggagtgtggtttctttttgaatctttaattaaaacctgggattatatatccctgataaatattcacacttgaaccatagttactgtaaaatgcaaaaaatcttaatactgttattctttgcactttttcttaatcattttttatatatatgcatatatatatgtgtgtgtgtgtgttgcttatgttgttttgtacagatgtgggccaccattgcaacaaaatacattctttttgctctaaaatatttatgaagaaaatacttaaatgttatgtatatggtggtaataagggaaaaatcaagtattataaacaagaatgaaggtttttgtaaagatttctgttcagcgttttgcaaggtaaaattttaggcaagttttccctgaagttatgtgtatgtgagtattctcattcttcccaacttgcctttgaagagtgaaataccattattatcaagtagactactgttcagcttttattcctgccctgctgtttatcccttaagaatgagtttcttagacttttccaatatgtgattttttttcccatttagaatggtgattttaaatgtgtgagtgcatgtactatcttatctcagatatttgcacccccaatctgcccccaactcccaaaagctagaacactgccaactgatctgttataggtcctttagaaacacataattaacacttaaggttgggtgctgctaattctttgcaaaaatccaaatattgttaagggaccagggagatgccactaccccttgattttccatctaaaaatatacatgtttatgtaaacaaatctttccatatccatagtgacttttcaagtatttaagcctaaagattttgatctcacatttttatacctgtttaaattgctcacagttattacatacacatcagccatcaactaaagttgtactttaaaaatttactacaatatgtacatttctaagtcaaacacttgtgacttttgctttaattccatgaatgttcctgcctccttgatatttgtatttattctttttttctctagagtagaggtataattgtgtgatatttcagaaatacagataaatgattcaaaaagtcacagttaaggagaatcatgtttctttgatcatgaataactgattagtaagtcttgcctatattttcctgatagcatatgacaaatgtttctaaggtaacaagatgagaacagataaagattgtgtggtgttttggatttggagagaaatattttaatttttaaatgcagttacaaattataatgtattcatatttgtactttctgttaaaatgcatgattgcagaattgtttagattttgtgtttattcttgatgaaaagctttgtttgttcttgtttttaagtttgcactcaaatcttaagaaataaatccacccatgttatcaaaaaaaaaaaaaaaaaa

SEQ ID NO: 4 - protein, 711 aa, Homo sapiens
MKLKLNVLTIILLPVHLLITIYSALIFIPWYFLTNAKKKNAMAKRIKAKPTSDKPGSPYRSVTHFDSLAVIDIPGADTLDKLFDHAVSKFGKKDSLGTREILSEENEMQPNGKVFKKLILGNYKWMNYLEVNRRVNNFGSGLTALGLKPKNTIAIFCETRAEWMIAAQTCFKYNFPLVTLYATLGKEAVVHGLNESEASYLITSVELLESKLKTALLDISCVKHIIYVDNKAINKAEYPEGFEIHSMQSVEELGSNPENLGIPPSRPTPSDMAIVMYTSGSTGRPKGVMMHHSNLIAGMTGQCERIPGLGPKDTYIGYLPLAHVLELTAEISCFTYGCRIGYSSPLTLSDQSSKIKKGSKGDCTVLKPTLMAAVPEIMDRIYKNVMSKVQEMNYIQKTLFKIGYDYKLEQIKKGYDAPLCNLLLFKKVKALLGGNVRMMLSGGAPLSPQTHRFMNVCFCCPIGQGYGLTESCGAGTVTEVTDYTTGRVGAPLICCEIKLKDWQEGGYTINDKPNPRGEIVIGGQNISMGYFKNEEKTAEDYSVDENGQRWFCTGDIGEFHPDGCLQIIDRKKDLVKLQAGEYVSLGKVEAALKNCPLIDNICAFAKSDQSYVISFVVPNQKRLTLLAQQKGVEGTWVDICNNPAMEAEILKEIREAANAMKLERFEIPIKVRLSPEPWTPETGLVTDAFKLKRKELRNHYLKDIERMYGGK